(19) United States
(12) Patent Application Publication
(10) Pub. No.: US 2004/0156832 A1
(43) Pub. Date: Aug. 12, 2004

(54) Immunoglobulin compositions and methods

(75) Inventor: Chris Jolly, Stanmore (AU)

Correspondence Address: SPECKMAN LAW GROUP PLLC, 1501 WESTERN AVE, SEATTLE, WA, 98101, US

(73) Assignees: CENTEC LIMITED, Newtown (AU); CENTENARY INSTITUTE OF CANCER MEDICINE AND CELL BIOLOGY, Camperdown (AU)

(21) Appl. No.: 10/672,764

(22) Filed: Sep. 26, 2003

Related U.S. Application Data
(60) Provisional application No. 60/415,024, filed on Sep. 30, 2002.

(51) Int. Cl. A61K 48/00; C12N 15/85; C12N 5/08

(52) U.S. Cl. 424/93.21; 435/455; 435/372

(57) ABSTRACT

Provided are methods for the preparation of immunoglobulins, including fully-human immunoglobulins, and immunoglobulins and compositions comprising immunoglobulins prepared by these methods. Also provided are libraries of immunoglobulins and immunoglobulin expressing cells as well as methods and vector constructs for preparation of these libraries. Immunoglobulins provided herein are useful inter alia as immunological and diagnostic agents and as therapeutic molecules in the treatment of diseases such as autoimmune diseases, heart disease, infections and cancers.

### CROSS-REFERENCE TO RELATED APPLICATIONS

This application claims priority to U.S. Provisional Patent Application No. 60/415,024, filed Sep. 30, 2002.

### REFERENCE TO SEQUENCE LISTING SUBMITTED ON COMPACT DISC

This application incorporates by reference in its entirety the Sequence Listing contained in the accompanying two compact discs, one of which is a duplicate copy. Each CD contains the following file: 1001U SEQLIST.txt, having a date of creation of Sep. 26, 2003 and constituting 1.64 MB (361 pages).

### BACKGROUND OF THE INVENTION

1. Technical Field of the Invention

The present invention relates generally to the fields of immunology and molecular biology. More specifically, this invention relates to methods for the preparation of immunoglobulins, including fully-human immunoglobulins, and to immunoglobulins and compositions comprising immunoglobulins prepared by the methods of the present invention. This invention also relates to libraries of immunoglobulins and immunoglobulin expressing cells as well as to methods and vector constructs for the preparation of these libraries. Immunoglobulins presented herein are useful, inter alia, as immunological and diagnostic agents and as therapeutic molecules in the treatment of diseases such as autoimmune diseases, heart disease, infections, and cancers.

2. Description of the Related Art

Conventional methodologies for the production of monoclonal antibodies (mAbs) generally involve the repeated immunization of rodents (usually mice) with an antigen of interest, isolation of antibody-producing B cells from killed immunized animals, and immortalization of the antibody-producing B cells by fusion with myeloma cells to generate B cell “hybridomas.” Libraries of hybridoma cells are screened for antigen-binding specificity and suitable clones purified and propagated.

As the fusion process is inefficient, potentially useful clones are frequently lost. More importantly, in terms of their potential clinical use, rodent derived mAbs are often antigenic when administered to humans. Consequently, a major limitation in the clinical use of rodent mAbs is the anti-globulin response. Miller et al. Blood 62:988-995, (1983); and Schroffet al. Cancer Res. 45:879-885 (1985).

Attempts to overcome this problem have been made by constructing chimeric antibodies in which an animal (non-human) antigen binding domain is coupled to a human constant domain. Morrison et al. Proc. Nat. Acad. Sci. USA 81:6851-6855 (1984); Boulianne et al. Nature 312:643-646 (1984); and Neuberger et al. Nature 314:268-270 (1985). Typically, chimeric antibodies contain approximately 33% non-human (normally rodent) protein and, therefore, are capable of eliciting a significant anti-globulin response in humans. For example, much of the anti-globulin response to mAb OKT3 is directed against the variable region of the molecule. Jaffers et al. Transplantation 41:572-578 (1986).

To further reduce the response to foreign protein, “humanized” mAbs have been produced. Jones et al., Nature 321:522-525 (1986); Reichmann et al., Nature 332:323-327 (1988); and Verhoeyen et al., Science 239:1534-1536 (1988). These mAbs are approximately 90-95% human with only the complementarity determining regions (CDRs) being non-human. Humanized mAbs produced in rodent cells are less immunogenic than chimeric mAbs. They still, however, can elicit an anti-globulin response in patients. Bell et al., Lancet 355:858-859 (2000). Fully-human antibodies are currently being generated through phage display and transgenic mouse methodologies. DNA fragments encoding antibody scFv fragments identified by phage display technology must be combined through recombinant techniques to generate complete “human” antibodies. To generate antibodies from transgenic mice, hybridomas must be prepared and screened as for conventional monoclonal antibody methodology.

Accordingly, there remains a need in the art for improved methods for the preparation of immunoglobulins that overcome these deficiencies in existing methodologies for the preparation of immunoglobulins, including fully-human immunoglobulins.

### SUMMARY OF THE INVENTION

The present invention addresses these and other related needs by providing, inter alia, methods for generating immunoglobulins including immunoglobulin heavy and/or light chains and fragments thereof. Also provided are methods for generating libraries of cells that produce arrays of immunoglobulins, and methods for identifying cells expressing immunoglobulins having desired antigen specificity. Further provided are immunoglobulins, including immunoglobulin heavy and/or light chains and fragments thereof, cell lines and libraries generated by the methods of the present invention. Still further provided are vector systems that encode various regions of immunoglobulins and vector systems encoding, various rearrangement-facilitating proteins.

Thus, provided herein are methods for generating immunoglobulin heavy and/or light chains, the methods comprising the steps of: (1) reverting a V(D)J rearranged immunoglobulin gene in a cell by introducing into the cell a polynucleotide encoding V, D, and/or J regions of an immunoglobulin heavy and/or light chain, or fragment thereof, wherein the V, D, and/or J regions replace the V(D)J rearranged immunoglobulin gene such that the introduced V, D, and/or J regions are in a pro-B cell-like or a germline-like state; and (2) expressing in the V, D, and/or J region reverted cell a polynucleotide sequence encoding a recombination-facilitating protein, or functional fragment, derivative or variant thereof, for a time and under conditions sufficient to induce rearrangement of the V, D, and/or J regions, wherein rearrangement of the V, D, and/or J regions facilitates expression of an immunoglobulin heavy and/or light chain.

Certain related embodiments provide methods for generating immunoglobulin heavy chains, the methods comprising the steps of: (1) reverting a V(D)J rearranged immunoglobulin gene in a cell by introducing into the cell a polynucleotide encoding fused DJ regions of an immunoglobulin heavy chain, wherein the DJ regions replace the V(D)J rearranged immunoglobulin gene such that the introduced fused DJ regions are in a pro-B cell-like state; and (2) expressing in the reverted cell a polynucleotide sequence encoding a recombination-facilitating protein, or functional fragment thereof, for a time and under conditions sufficient to induce rearrangement of the germline V regions in the reverted cell with the introduced fused DJ regions, wherein rearrangement of the V and fused DJ regions facilitates expression of an immunoglobulin heavy chain.

Other related embodiments provide methods for generating immunoglobulin light chains, the methods comprising the steps of: (1) reverting a V(D)J rearranged immunoglobulin gene in a cell by introducing into the cell a polynucleotide encoding J regions of an immunoglobulin light chain, wherein the J regions replace the V(D)J rearranged immunoglobulin gene such that the introduced J regions are in a pro-B cell-like or a germline-like state; and (2) expressing in the reverted cell a polynucleotide sequence encoding a recombination-facilitating protein, or functional fragment thereof, for a time and under conditions sufficient to induce rearrangement of the germline V regions in the reverted cell with the introduced J regions, wherein rearrangement of the V and J regions facilitates expression of an immunoglobulin light chain.

Within other aspects of the present invention are provided methods for generating libraries of cells that produce an array of immunoglobulins wherein each immunoglobulin exhibits a particular antigen specificity. Exemplary methods comprise the steps of: (1) providing a cell having a V(D)J rearranged immunoglobulin heavy and/or light chain gene; (2) introducing into the cell a polynucleotide encoding V, D, and/or J regions of an immunoglobulin heavy and/or light chain, or fragment thereof, wherein said V, D, and/or J regions replace said V(D)J rearranged immunoglobulin gene such that the introduced V, D, and/or J regions are in a pro-B cell-like or a germline-like state; (3) culturing the cell under suitable conditions to generate a population of cells each member of which population comprises V, D, and/or J regions in a pro-B cell-like or a germline-like state; (4) introducing into cells of the reverted cell population a polynucleotide sequence encoding a recombination-facilitating protein, or functional fragment, derivative or variant thereof; and (5) culturing the resulting population of cells expressing the recombination-facilitating protein for a time and under conditions sufficient to induce rearrangement of the pro-B cell-like or germline-like V, D, and/or J regions, wherein rearrangement of the V, D, and/or J regions facilitates expression of an immunoglobulin heavy and/or light chain having a particular antigen specificity.

Other aspects provide methods for identifying in a cell an immunoglobulin having a desired antigen specificity, the methods comprising the steps of: (1) reverting a V(D)J rearranged immunoglobulin gene in a cell by introducing into the cell a polynucleotide encoding V, D, and/or J regions of an immunoglobulin heavy and/or light chain, or fragment thereof, wherein the V, D, and/or J regions replace the V(D)J rearranged immunoglobulin gene such that the introduced V, D, and/or J regions are in a pro-B cell-like or a germline-like state; (2) expressing in the reverted cell a polynucleotide sequence encoding a recombination-facilitating protein, or functional fragment, derivative, or variant thereof, for a time and under conditions sufficient to induce rearrangement of the pro-B cell-like or germline-like V, D, and/or J regions, wherein rearrangement of the V, D, and/or J regions facilitates expression of an immunoglobulin heavy and/or light chain; and (3) screening the resulting V, D, and/or J region rearranged cells for an immunoglobulin having the desired antigen specificity.

Within other related aspects, the present invention provides methods for generating cell lines capable of producing immunoglobulins having a desired specificity, the methods comprising the step of reverting a V(D)J rearranged immunoglobulin gene in a cell by introducing into the cell a polynucleotide encoding V, D, and/or J regions of an immunoglobulin heavy and/or light chain, or fragment thereof, wherein the V, D, and/or J regions replace the V(D)J rearranged immunoglobulin gene such that the introduced V, D, and/or J regions are in a pro-B cell-like or a germline-like state.

Still further related aspects provide methods for generating cell lines capable of producing immunoglobulins having a desired specificity, the methods comprising the step of reverting a V(D)J rearranged immunoglobulin gene in a cell by introducing into the cell a polynucleotide encoding one or more fused DJ regions of an immunoglobulin heavy chain, wherein the DJ regions replace the V(D)J rearranged immunoglobulin gene such that the introduced fused DJ regions are in a pro-B cell-like state. Within certain methods, the introduced polynucleotide comprises two or more fused DJ regions. Alternative methods provide that the polynucleotide comprises at least three, four, or five fused DJ regions. Still further methods provide that the polynucleotide comprises six fused DJ regions. An exemplary polynucleotide comprising six fused DJ regions is presented herein in FIG. 1.

Other aspects of the present invention provide methods for producing immunoglobulins having a particular affinity or specificity for a target molecule, the methods comprising the steps of: (1) generating a reverted lymphoma cell line capable of producing immunoglobulins; (2) expressing a polynucleotide encoding a recombination-facilitating protein, or functional fragment, derivative, or variant thereof, in the reverted lymphoma cell line for a time and under conditions sufficient to induce a rearrangement of the genes encoding the immunoglobulins which facilitates the generation of a library of lymphoma cells which produce an array of immunoglobulins wherein each immunoglobulin exhibits a particular affinity or specificity; and (3) screening for immunoglobulins having a desired affinity or specificity.

Within other aspects of the present invention are provided methods for producing libraries of human monoclonal antibody-producing lymphoma cells, the methods comprising the step of expressing in a reverted human lymphoma cell a polynucleotide encoding a recombination-facilitating protein for a time and under conditions sufficient to induce rearrangement of genes encoding human antibodies in the reverted lymphoma cells.

In other aspects, the present invention provides methods for generating a library of lymphoma cell lines of human origin, each human lymphoma cell line capable of producing filly-human immunoglobulins of a particular specificity, the method comprising the steps of: (1) reverting a V(D)J rearranged immunoglobulin gene in a human lymphoma cell line by introducing a polynucleotide encoding human V, D, and/or J regions of human immunoglobulin heavy and/or light chains into the human lymphoma cell line wherein the V, D, and/or J regions replace the V(D)J rearranged immunoglobulin gene, wherein the V, D, and/or J regions replace said V(D)J rearranged immunoglobulin gene such that the introduced V, D, and/or J regions are in a pro-B cell-like or germline-like state; (2) expressing in the reverted human lymphoma cell a polynucleotide sequence encoding a human recombination-facilitating protein, or a functional fragment, derivative or variant thereof, for a time and under conditions sufficient to facilitate rearrangement of immunoglobulin-encoding genes thus generating a library of human lymphoma cells each producing a filly-human immunoglobulin of a particular specificity.

In certain related aspects, the present invention provides methods for generating a library of lymphoma cell lines of human origin, each human lymphoma cell line capable of producing fully-human immunoglobulin heavy chains of a particular specificity, the method comprising the steps of: (1) reverting a V(D)J rearranged immunoglobulin gene in a human lymphoma cell line by introducing a polynucleotide encoding fused DJ regions of a human immunoglobulin heavy chain into the cell line, wherein the DJ regions replace the V(D)J rearranged immunoglobulin gene such that the introduced fused DJ regions are in a pro-B cell-like state; (2) expressing in the reverted human cell a polynucleotide sequence encoding a human recombination-facilitating protein, or functional fragment, derivative or variant thereof, for a time and under conditions sufficient to induce rearrangement of the human lymphoma cell line germline V regions with the introduced fused DJ regions, wherein rearrangement of the V and fused DJ regions facilitates expression of a fully-human immunoglobulin heavy chain. An exemplary polynucleotide encoding fused DJ regions is presented herein in FIG. 1.

Still further related aspects provide methods for generating a library of lymphoma cell lines of human origin, each human lymphoma cell line capable of producing fully-human immunoglobulin light chains of a particular specificity, the method comprising the steps of: (1) reverting a V(D)J rearranged immunoglobulin gene in a human lymphoma cell by introducing a polynucleotide encoding J regions of a human immunoglobulin light chain into the human lymphoma cell line, wherein the J regions replace the V(D)J rearranged immunoglobulin gene such that the introduced J regions are in a pro-B cell-like or a germline-like state; (2) expressing in the reverted human lymphoma cell line a polynucleotide sequence encoding a human recombination-facilitating protein, or functional fragment, derivative or variant thereof, for a time and under conditions sufficient to induce rearrangement of the germline V regions with the introduced J regions, wherein rearrangement of the V and J regions facilitates expression of a fully-human immunoglobulin light chain.

In those embodiments of the present invention wherein cells containing V(D)J rearranged immunoglobulin genes are reverted to a pro-B cell-like state, reversion is achieved by introducing a vector, such as a plasmid vector, comprising a polynucleotide encoding fused DJ regions of an immunoglobulin heavy chain, or fragment thereof. Alternatively, cells containing V(D)J rearranged immunoglobulin genes may be reverted to a germline-like state by introducing a vector comprising a polynucleotide encoding V, D, and/or J regions of immunoglobulin heavy and/or light chains, or fragments thereof, wherein the V, D, and/or J regions are assembled in the vector in a germline-like state.

Thus, the present invention provides vectors, including plasmid vectors, useful in reverting cell lines to a pro-B cell-like state or to a germline-like state, the vectors comprising one or more immunoglobulin regions including V regions, D regions, and/or J regions as well as any combination thereof. More preferred vectors comprise D and/or J regions. Still more preferred are vectors for reverting cell lines to a pro-B cell-like state wherein such vectors comprise fused DJ regions that approximate a rearranged form.

Vectors of the present invention further comprise a 5′ flanking region and a 3′ flanking region for facilitating homologous recombination of the antibody regions into a cell having a rearranged immunoglobulin gene. Generally, the 5′ flanking region (a) comprises a promoter region, such as a VH promoter region, and (2) is operably linked 5′ to the 5′-most fused DJ region, and the 3′ flanking region is operably linked 3′ to the 3′-most fused DJ region. Optionally, the vector may further comprise a selectable marker gene 5′ to the 5′-most fused DJ region and 3′ to the 5′ flanking region. Within certain embodiments, expression of the selectable marker is regulated by a promoter, such as the VH promoter, contained within the 5′ flanking region.

Each of the vectors disclosed herein facilitate replacement of the endogenous V(D)J rearrangement by introducing V, D, and/or J regions by homologous recombination. An exemplary plasmid vector for introducing fused DJ regions by homologous recombination is presented herein in FIG. 1.

In those embodiments of the present invention wherein cells containing V(D)J rearranged immunoglobulin genes are reverted to a germline-like state, reversion may be achieved by introducing a chromosome, or substantial portion thereof, comprising a sequence encoding V, D, and/or J regions of an immunoglobulin heavy and/or light chain, or fragment thereof wherein the chromosome encodes a complete germline heavy or light chain antibody repertoire or substantial portion thereof. Preferred chromosomes include human chromosomes 2, 14, and 22. In the most preferred embodiments, the introduced chromosome replaces the portion of the chromosome that corresponds to V(D)J rearrangements in chromosome 2, 14, and/or 22, respectively.

Chromosomes may be introduced into a cell by a methodology including, but not limited to, microcell-mediated chromosome transfer, T cell-fusion, micro-injection, and/or yeast protoplast fusion. Within certain aspects, cells containing V(D)J rearranged immunoglobulin genes are reverted to a germline-like configuration by fusing a B cell line with precursor B cells isolated from human bone marrow or cord blood. Other aspects provide that cells may be reverted to a germline-like configuration by fusing a B cell line with T cells isolated from human blood. Still further aspects provide that cells are reverted to a germline-like configuration by fusing B cell lines with rodent/human somatic cell hybrids carrying single human chromosomes.

Certain aspects of the present invention are directed to the generation of immunoglobulins of human origin. The presently disclosed methods may also be employed for the production of non-human immunoglobulins of primate, sheep, pig, cow, horse, donkey, poultry, rabbit, mouse, rat, guinea pig, hamster, dog, and cat origin.

Recombination-facilitating proteins, or functional fragments, derivatives or variants thereof, that are suitable in any of the methods disclosed herein may be selected from the group consisting of RAG-1 and RAG-2. RAG-1 and RAG-2 proteins may be from any species including primates, livestock animals, laboratory test animals and companion animals as well as functionally similar molecules from avian, reptilian, amphibian or aquatic animals. More preferred are RAG-1 and RAG-2 proteins from humans such as those provided herein as SEQ ID NOs: 2 and 4.

Recombination-facilitating proteins, or functional fragments, derivatives or variants thereof, may be expressed transiently for a time and under conditions sufficient to achieve recombination in any of the reverted cells recited herein. Within other embodiments, the recombination-facilitating proteins may be expressed constitutively and expression of these proteins may be under the control of an inducible transcriptional promoter.

Preferred cells to be employed in the methods of the present invention include any immunoglobulin-producing cell line such as, for example, lymphocytes and lymphocyte cell lines such as B lymphocytes, B lymphocyte cell lines, B cell lymphoma cells, and B cell lymphoma cell lines. More preferred are human lymphoma cell lines including, but not limited to, human B cell lines generated from patients with Burkitt's lymphoma. Most preferred cell lines are Ramos, Ramos sub-line 2G6, Burkitt's lymphoma cell lines BL2 and BL16 and BL16 sub-line CL-01.

Other embodiments of the present invention provide immunoglobulins and fragments thereof, including, but not limited to Fab, F(ab′)₂, Fc, scFv that are generated by any of the methods disclosed herein.

Further aspects of the present invention provide that cells expressing immunoglobulins and fragments thereof, generated by any of the methods presented herein may be further treated with an agonist, or combination of agonists, to induce switching from a first antibody isotype to a second antibody isotype. Within these aspects, the first antibody isotype may be selected from the group consisting of IgM, IgD, IgG1, IgG2, IgG3, IgG4, IgE, IgA1 and IgA2; the second antibody isotype may be selected from the group consisting of IgD, IgG1, IgG2, IgG3, IgG4, IgE, IgA1 and IgA2.

Suitable agonists include, but are not limited to, (1) ligands for CD40 such as CD154, anti-CD40 or fresh T cells activated with, for instance, phytohaemoglutenin; (2) ligands for the B cell receptor such as anti-Ig or anti-Ig coupled to dextran; (3) B cell mitogens such as purified protein derivative from Mycobacterium species (PPD), or bacterial DNA, or synthetic oligonucleotides containing unmethylated CpG dinucleotides; (4) cytokines such as IL2, IL4, IL5, IL6, IL10, IL13, TGFβ or IFNγ; (5) anti-CD19; and/or (6) anti-CD21. Cells expressing immunoglobulins may be exposed to one or more of these agonists alone or in combination.

Within certain aspects, cells and/or libraries of cells expressing one or more immunoglobulin, or fragment(s) thereof, may be further subjected to mutagenesis to, for example, increase efficiency of antibody production and/or increase efficiency of binding to, and/or neutralizing, target molecules of interest. Exemplary methods for achieving mutagenesis comprise the step of introducing into the cell and/or library of cells a polynucleotide encoding activation-induced cytidine deaminase (AICD). Thus, certain methods comprise the step of introducing into the cell and/or library of cells a polynucleotide encoding human activation-induced cytidine deaminase (AICD) provided herein as SEQ ID NO: 38.

Further aspects of the present invention provide that the antigen specificity and/or affinity of immunoglobulins generated by any of the methods presented herein may be altered by introducing into the cells and/or libraries of cells a polynucleotide encoding Terminal Deoxynucleotidyltransferase (TdT) or a functional fragment, derivative or variant thereof. Most preferred is human TdT as disclosed herein in SEQ ID NO: 6. Preferably, the polynucleotide encoding TdT is introduced into the cells and/or library of cells coincident with the introduction of the polynucleotide(s) encoding the recombination-facilitating protein.

Still further embodiments of the present invention provide vectors for expressing recombination-promoting proteins and fragments, derivatives, and variants thereof Preferred vectors comprise one or more polynucleotide sequences encoding recombinant-promoting proteins including, but not limited to, RAG-1 and/or RAG-2. More preferred are vectors comprising one or more polynucleotide sequence encoding human RAG-1 and/or RAG-2 as disclosed herein in SEQ ID NOs: 1 and 3. Exemplary vectors are presented herein in FIGS. 2, 5B (pBI-TdT-GFP.RAG2, SEQ ID NO: 42), and SEQ ID NO: 40 (pcDNA3-RAG1) and are described in further detail within the Examples herein below.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS AND SEQUENCE IDENTIFIERS

FIG. 1 is a diagrammatic representation of an exemplary recombinant DNA construct useful for reverting, in a cell, rearranged immunoglobulin genes to a pro-B cell-like state. Each fragment of the construct was generated by PCR using the primers and DNA templates indicated. Fragments were ligated together after digestion of the PCR products with the restriction enzymes indicated.

FIG. 2 is a diagrammatic representation of an adenovirus vector for expressing Rag-2, useful for facilitating rearrangement of immunoglobulin regions that are in a pro-B cell like or a germline-like state.

FIGS. 3A through 3D depict the analysis of G418 plus hygromycin double-resistant RK1 and RK2 cell lines generated by microcell-mediated chromosome transfer (MMCT) from A9-Hytk14 cells and G418-resistant Ramos cells. FIG. 3A is a photograph of an agarose gel showing the detection of J_{H} elements normally absent from Ramos in double-resistant lines. DNA extracted from A9-Hytk14, Ramos, RK1.1, and RK1.2 cell lines was subjected to PCR using primer A (that can anneal to all six J_{H} elements, SEQ ID NO: 43) paired with one of primers B to G (specific for sequences downstream of each individual J_{H} element, SEQ ID NOs: 44-49, respectively). FIG. 3B is a graph of fluorescence activated cell sorting (FACS) data obtained from cells stained with anti-Igκ conjugated to FITC. FIG. 3C is a graph of FACS data obtained from cells stained for total (surface+intracellular) IgM expression by RK2.1/x and RK2.2/x lines (x=1 to 5) which were cloned by limiting dilution from surface Ig-depleted RK1.1 and RK1.2 cells, respectively. RK1.1 and RK1.2 cells depleted of surface Ig-positive cells (e.g., FIG. 3B) were cloned by limiting dilution. FIG. 3D is a photograph of an agarose gel showing the absence of the rearranged V(D)J_{H} gene segment in some surface Ig-depleted Ramos-glH sub-clones.

FIGS. 4A through 4C are immunoblots demonstrating the detection of RAG1 and TdT proteins. RAG1 protein was readily detectable in unmanipulated Ramos cells (FIG. 4A). TdT protein was not detectable in Ramos cells (FIG. 4B) until transduced with TdT-expressing plasmid (FIG. 4C) or adenovirus (data not shown).

FIGS. 5A through 5C present plasmid maps of pIRES-rtTA-puro (FIG. 5A, SEQ ID NO: 41) and pBI-TdT-GFP.RAG2 (FIG. 5B, SEQ ID NO: 42) (described in the Examples presented herein below) and a histogram of FACS data demonstrating doxycycline-regulated expression of GFP-RAG2 and TdT (FIG. 5C). Plasmids pIRES-rtTA-puro and pBI-TdT-GFP.RAG2 were linearized and co-transfected into RK2.1/4 cells and RK2.2/3 cells. Puromycin-resistant colonies were exposed to doxycycline for 7 days. Peak GFP-fluorescence was detected on day 4 as shown for the exemplary RK2.2/3 transfectant designated RK6.6. FIG. 5C.

FIG. 6 presents a histogram of, and table quantifying, FACS data of GFP expression in cells transiently infected with non-replicating recombinant GFP-adenovirus. Histograms show fluorescence due to GFP expression in Ramos cells, NIH 3T3 cells, RD1 cells—Ramos cells transfected with pCAR-IRES-neo, and Ramos cells previously sorted by FACS as a denovirus-susceptible. The timepoint of maximal GFP fluorescence is shown—day 3 for Ramos lines and day 1 for NIH 3T3 cells. The table indicates the relative frequencies of cells in infected cultures expressing GFP. In all cases fluorescence almost disappeared by day 5.

FIGS. 7A through 7D present FACS data obtained at various points during the purification of phOx antigen-specific cells from Ramos cultures. Flow cytometric analyses of the frequency of phOx antigen-specific cells present after each enrichment step are shown. FITC fluorescence indicates cells that bind BSA conjugated to both FITC and phOx. A culture of 10⁸ Ramos cells was seeded with 100 phOx-specific “transfectoma” (RCC64) cells to give a precursor frequency of 1 in 10⁶. FIG. 7A. Selection for phOx-binding cells using MACS beads led to a 600-fold enrichment for phOx-specific Ramos cells. FIGS. 7A and 7B. Further selection by 2-step flow cytometric sorting led to a total enrichment of 10⁶-fold to give a purity of 96%. FIGS. 7B through 7D.

SEQ ID NO: 1 is a cDNA sequence encoding the human RAG-1 protein sequence of SEQ ID NO: 2 (Genbank Accession No. NM_{—}000448).

SEQ ID NO: 2 is the human RAG-1 protein sequence encoded by the cDNA sequence of SEQ ID NO: 1 (Genbank Accession No. NP_{—}000439).

SEQ ID NO: 3 is a cDNA sequence encoding the human RAG-2 protein sequence of SEQ ID NO: 4 (Genbank Accession No. XM_{—}089839).

SEQ ID NO: 4 is the human RAG-2 protein sequence encoded by the cDNA sequence of SEQ ID NO: 3 (Genbank Accession No. XP_{—}089839).

SEQ ID NO: 5 is a cDNA sequence encoding the human Terminal Deoxynucleotidyltransferase (TdT) protein sequence of SEQ ID NO: 6 (Genbank Accession No. XM_{—}055459).

SEQ ID NO: 6 is the human Terminal Deoxynucleotidyltransferase (TdT) protein sequence encoded by the cDNA sequence of SEQ ID NO: 5 (Genbank Accession No. XP_{—}055459).

SEQ ID NO: 7 is an oligonucleotide primer sequence designated herein as 5′ VH4-34 (KpnI).

SEQ ID NO: 8 is an oligonucleotide primer sequence designated herein as 3′ VH4-34 (ApaI).

SEQ ID NO: 9 is an oligonucleotide primer sequence designated herein as 5′ JH63 'fl (NotI).

SEQ ID NO: 10 is an oligonucleotide primer sequence designated herein as 3′ JH63 'fl (SacII).

SEQ ID NO: 11 is an oligonucleotide primer sequence designated herein as 3′ SnaBI (VH4-34).

SEQ ID NO: 12 is an oligonucleotide primer sequence designated herein as 5′ SnaBI (VH4-34).

SEQ ID NO: 13 is an oligonucleotide primer sequence designated herein as 5′ Eco-gpt.

SEQ ID NO: 14 is an oligonucleotide primer sequence designated herein as 3′ Eco-gpt.

SEQ ID NO: 15 is an oligonucleotide primer sequence designated herein as 5′ D3-10.1.

SEQ ID NO: 16 is an oligonucleotide primer sequence designated herein as 3′ JH1.

SEQ ID NO: 17 is an oligonucleotide primer sequence designated herein as 5′ D3-10.2.

SEQ ID NO: 18 is an oligonucleotide primer sequence designated herein as 3′ JH2.

SEQ ID NO: 19 is an oligonucleotide primer sequence designated herein as 5′ D3-10.3.

SEQ ID NO: 20 is an oligonucleotide primer sequence designated herein as 3′ JH3.

SEQ ID NO: 21 is an oligonucleotide primer sequence designated herein as 5′ D3-10.4.

SEQ ID NO: 22 is an oligonucleotide primer sequence designated herein as 3′ JH4.

SEQ ID NO: 23 is an oligonucleotide primer sequence designated herein as 5′ D3-10.5.

SEQ ID NO: 24 is an oligonucleotide primer sequence designated herein as 3′ JH5.

SEQ ID NO: 25 is an oligonucleotide primer sequence designated herein as 5′ D3-10.6.

SEQ ID NO: 26 is an oligonucleotide primer sequence designated herein as 3′ JH6.

SEQ ID NO: 27 is the nucleotide sequence of the gpt gene in cloning vector pSV2 gpt (Genbank Accession No. M12907).

SEQ ID NO: 28 is the amino acid sequence of the Gpt protein (Genbank Accession No. AAA23932) encoded by the gpt gene in cloning vector pSV2.

SEQ ID NO: 29 is the nucleotide sequence for a human DNA for immunoglobulin alpha heavy chain (Genbank Accession No. X17116).

SEQ ID NO: 30 is the nucleotide sequence for Homo sapiens immunoglobulin heavy-chain variable region (Genbank Accession No. AB019437).

SEQ ID NO: 31 is the nucleotide sequence for Homo sapiens immunoglobulin heavy-chain variable region (Genbank Accession No. AB019438).

SEQ ID NO: 32 is the nucleotide sequence for Homo sapiens immunoglobulin heavy-chain variable region (Genbank Accession No. AB019439).

SEQ ID NO: 33 is the nucleotide sequence for Homo sapiens immunoglobulin heavy-chain variable region (Genbank Accession No. AB019440).

SEQ ID NO: 34 is the nucleotide sequence for Homo sapiens immunoglobulin heavy-chain variable region (Genbank Accession No. AB019441).

SEQ ID NO: 35 is the nucleotide sequence for Homo sapiens RhD blood group antigen cDNA (Genbank Accession No. L08429).

SEQ ID NO: 36 is the nucleotide sequence for an oligonucleotide primer useful in PCR amplifying cDNA encoding human Rag-2.

SEQ ID NO: 37 is the nucleotide sequence for an oligonucleotide primer useful in PCR amplifying cDNA encoding human Rag-2.

SEQ ID NO: 38 is a cDNA sequence encoding the human activation-induced cytidine deaminase protein depicted in SEQ ID NO: 40 (Celera transcript hCT16345).

SEQ ID NO: 39 is the human activation-induced cytidine deaminase protein sequence encoded by the cDNA sequence of SEQ ID NO: 39 (Celera protein hCP42155).

SEQ ID NO: 40 is nucleotide sequence of the plasmid CJ087: pCDNA3-RAG1.

SEQ ID NO: 41 is the nucleotide sequence of the plasmid pIRES-rtTA-puro.

SEQ ID NO: 42 is the nucleotide sequence of the plasmid pBI-TdT-GFP.RAG2.

SEQ ID NO: 43 is the nucleotide sequence of primer “A” (5′-GGT CAC CGT CTC YTC AGG T-3′).

SEQ ID NO: 44 is the sequence of oligonucleotide primer “B” (5′-GAT ATC GAT ACC AGT AGC ACA GCC TCT G-3′).

SEQ ID NO: 45 is the sequence of oligonucleotide primer “C” (5′-CTG GAA TTC TGC AGG ACA CTC GAA TGG-3′).

SEQ ID NO: 46 is the sequence of oligonucleotide primer “D” (5′-TGC GGA TCC ACC TGA CTC TCC GAC TGT CC-3′).

SEQ ID NO: 47 is the sequence of oligonucleotide primer “E” (5′-TA ACT AGT TGG GAC CCT CTC AGA CT-3′).

SEQ ID NO: 48 is the sequence of oligonucleotide primer “F” (5′-CA TCT AGA CAG AGA CCT TCT GTC TCC G-3′).

SEQ ID NO: 49 is the sequence of oligonucleotide primer “G” (5′-TGA GCG GGC CGC GGC CTC AAT TCC AGA CAC AT-3′).

SEQ ID NO: 50 is the sequence of oligonucleotide primer jo1209 (5′-ATG AAA CAC CTG TGG TTC TTC CTC C-3′).

SEQ ID NO: 51 is the sequence of oligonucleotide primer jo191 (5′-CGG GTA CCA ACC TGC AAT GCT CAG GA-3′).

SEQ ID NO: 52 is the nucleotide sequence of the plasmid pShuttle-RAG2.

SEQ ID NO: 53 is the nucleotide sequence of the plasmid pShuttle-GFP-RAG2.

SEQ ID NO: 54 is the nucleotide sequence of the plasmid pAdEasy-RAG2.

SEQ ID NO: 55 is the nucleotide sequence of the plasmid pAdEasy.1-GFP-RAG2.

SEQ ID NO: 56 is the nucleotide sequence of the plasmid pAdEasy.2-GFP-RAG2.

SEQ ID NO: 57 is the sequence of oligonucleotide primer “Jo1266” (5′-GAC TCT AGA GCA GCT CCA AAG ATG GCA TGC G-3′).

SEQ ID NO: 58 is the sequence of oligonucleotide primer “Jo1267” (5′-GTT TGA ATT CCA CCT TGG TCC CTT GG-3′).

SEQ ID NO: 59 is the sequence of oligonucleotide primer “kam8” (5′-CCT GCT CTG GAG ATA AAT TGG G-3′).

SEQ ID NO: 60 is the sequence of oligonucleotide primer “kam9” (5′-CTG CTG TCC CAC GCC TGA CA-3′).

SEQ ID NO: 61 is the sequence of oligonucleotide primer “Jo1272” (5′-CCA TCG ATG CCT ACC TGC AGC CGC CGC CC-3′).

SEQ ID NO: 62 is the sequence of oligonucleotide primer “Jo1273” (5′-CGG GAT CCG AGG CTC TAT ACT ATA GAC-3′).

SEQ ID NO: 63 is the sequence of the plasmid pCAR-IRES-neo (pCJ124).

SEQ ID NO: 64 is the sequence of the plasmid pCAR-IRES-puro (pCJ126).

SEQ ID NO: 65 is the sequence of oligonucleotide primer “Jo1289” (5′-GG ATA TCG CTG CCC CCA AGT GTA ACT C-3′).

SEQ ID NO: 66 is the sequence of oligonucleotide primer “Jo1290” (5′-TAA AGC GGC CGC TTA CTT ATC GTC GTC ATC CTT GTA ATC AGG ATC CAT TGG TTC AAC TGT CTC-3′).

SEQ ID NO: 67 is the sequence of the plasmid pBlimp 1-IRES-bleo.

SEQ ID NO: 68 is the sequence of the plasmid pro-B IgH (PH017).

### DETAILED DESCRIPTION OF THE INVENTION

As indicated above, the present invention is directed to methods for the preparation of immunoglobulins, including fully-human immunoglobulins, and to immunoglobulins and compositions comprising immunoglobulins prepared by the methods of the present invention. Also provided herein are libraries of immunoglobulins and immunoglobulin expressing cells as well as methods and vector constructs for the preparation of these libraries. Immunoglobulins presented herein are useful, inter alia, as immunological and diagnostic agents and as therapeutic molecules in the treatment of diseases such as autoimmune diseases, heart disease, infections, and cancers.

As used in this specification and the appended claims, the singular forms “a,” “an” and “the” include plural references unless the content clearly dictates otherwise. The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al., “Molecular Cloning: A Laboratory Manual” (2nd Edition, 1989); Maniatis et al., “Molecular Cloning: A Laboratory Manual” (1982); “DNA Cloning: A Practical Approach, vol. I & II” (D. Glover, ed.); “Oligonucleotide Synthesis” (N. Gait, ed., 1984); “Nucleic Acid Hybridization” (B. Hames & S. Higgins, eds., 1985); “Transcription and Translation” (B. Hames & S. Higgins, eds., 1984); “Animal Cell Culture” (R. Freshney, ed., 1986); Perbal, “A Practical Guide to Molecular Cloning” (1984); Ausubel, et al. “Current protocols in Molecular Biology” (New York, John Wiley and Sons, 1987); Bonifacino, et al. “Current Protocols in Cell Biology” (New York, John Wiley & Sons, 1999); and Coligan, eta L. “Current Protocols in Immunology” (New York, John Wiley & Sons, 1999).

All publications, patents and patent applications cited herein, whether supra or infra, are hereby incorporated by reference in their entirety.

Reference herein to an “immunoglobulin” includes and is most preferably an immunoglobulin such as an antibody or biological or functional equivalent thereof. Reference herein to an “antibody” or the more generic term “immunoglobulin” includes reference to parts, fragments, precursor forms, derivatives, variants, and genetically engineered or naturally mutated forms thereof and includes amino acid substitutions and labeling with chemicals and/or radioisotopes and the like, so long as the resulting derivative and/or variant retains at least a substantial amount of antigen binding specificity and/or affinity.

Preferred mammalian immunoglobulins are human immunoglobulins such as human antibodies or, more particularly, human monoclonal antibodies. As used herein, the term “immunoglobulin” broadly includes both immunoglobulin heavy and light chains as well as all isotypes of antibodies, including IgM, IgD, IgG1, IgG2, IgG3, IgG4, IgE, IgA1 and IgA2, and also encompasses antigen binding fragments thereof, including, but not limited to, Fab, F(ab′)₂, Fc, and scFv.

The present invention is based on the finding that cells, including immunoglobulin expressing cells, that have recombined their immunoglobulin regions through the process of V(D)J joining, may be reverted to a pro-B cell-like and/or a germline-like state by introducing into the cell polynucleotides encoding one or more V, D, and/or J regions. As detailed herein, recombination of the immunoglobulin regions within the pro-B cell-like and/or germline-like reverted cell may be achieved by introducing into the cell a polynucleotide encoding one or more recombination facilitating protein such as, for example, RAG-1 and/or RAG-2. The affinity and/or specificity of immunoglobulins generated by the present methods may by altered by one or more mutagenesis steps such as introducing Terminal Deoxynucleotidyltransferase (TdT) into the cell, preferably simultaneous to introduction of a recombination-facilitating protein, or by constitutive or induced expressed of Activation-Induced Cytidine Deaminase (AICD).

As used herein, the term “pro-B cell-like” includes cells mimicking an early B cell lineage wherein rearrangement of D_{H} and J_{H} regions has already occurred. As described in greater detail and exemplified herein, a “pro-B cell-like” state may be achieved by introducing into a B- or plasma-cell and/or cell-line (e,g., Ramos) one or more fused DJ_{H} regions that mimic the in vivo DJ recombination event that occurs in the early pro-B cell stage, wherein the DJ_{H} regions replace the endogenous V(D)J_{H} rearranged immunoglobulin regions by homologous recombination.

Throughout the specification, the abbreviated designations V_{H}, D_{H}, and J_{H} are used interchangeably with the phrase heavy chain V, D, and J regions. Similarly, the abbreviated designations Vκ, Jκ, Vκ, and Jλ are used interchangeably with the phrases kappa light chain V and J regions and lambda light chain V and J regions, respectively. Also, the term “regions” has a meaning equivalent to the phrase “gene segment” when used in the context of V, D and/or J “regions” and “gene segments.” The terms DJ, V(D)J, and the like refer to a fused and/or rearranged form of V, D, and/or J regions. The term “germline-like” refers to cells mimicking a stage in the B cell lineage preceding the “pro-B cell-like” stage wherein each of the immunoglobulin gene segments are separated one from the other as is the case in immunoglobulin expressing cells before any in vivo recombination events occur.

As described and exemplified herein, a “germline-like” state may be achieved by introducing into a reverted cell one or more V, D, and/or J regions in a separate, unfused arrangement. A germline-like state may, for example, be achieved by introducing into the cell a vector comprising one or more V, D, and/or J region in a separate, unfused arrangement. Alternatively, introducing into the cell a whole chromosome, or substantial portion thereof may achieve a germline-like state, wherein the chromosome encodes a complete repertoire of germline immunoglobulin heavy or light chain region gene segments or substantial portion thereof.

The methods of the present invention will be better understood through the detailed description of the following specific embodiments:

(a) selection and/or generation of immunoglobulin expressing cells and cell-lines including B cell-lines, such as Burkitt's lymphoma (BL) cell-lines, that undergo isotype switching and somatic mutation either constitutively or inducibly;

(b) engineering of immunoglobulin expressing cells to revert their rearranged immunoglobulin genes to approximate a pro-B cell-like or a germline-like state;

(c) generation of libraries of immunoglobulin expressing cells by rearranging the immunoglobulin V, D, and/or J regions within the reverted cell-line, in a random manner mimicking in vivo B cell development, by introducing a polynucleotide encoding one or more recombination-facilitating protein including polypeptide components of a V(D)J-recombinase;

(d) enrichment and/or isolation of cells expressing immunoglobulins having a desired antigen affinity and/or specificity;

(e) mutation of cells that express an immunoglobulin having affinity and/or specificity for a target antigen by utilizing the innate ability of some immunoglobulin expressing cells and cell-lines to undergo somatic mutation and/or by introduction of a polynucleotide that encodes a mutagenesis promoting protein such as, for example, Activation-induced Cytidine Deaminase (AICD) and/or Terminal Deoxynucleotidyltransferase (TdT); and

(f) induction of immunoglobulin isotype switching by exposing immunoglobulin expressing cells to one or more cytokine and/or mitogen known to induce isotype switching in immunoglobulin expressing cells such as, for example, human B cell lymphoma cells.

Each of these embodiments is described in greater detail herein below.

### Selection of Immunoglobulin Expressing Cells and Cell-lines for Generating Antibodies

As indicated above, the present invention provides methods for generating immunoglobulins, including immunoglobulin heavy and/or light chains, as well as methods for generating cells and libraries of cells that express immunoglobulins having a desired antigen specificity and/or affinity. Each of the methods disclosed herein utilize a cell or cell line that is capable of expressing immunoglobulin genes. Therefore, certain preferred cells to be employed in the methods of the present invention include any immunoglobulin-producing cell or cell line such as, for example, lymphocytes and lymphocyte cell lines such as B lymphocytes, B lymphocyte cell lines, B cell lymphoma cells, and B cell lymphoma cell lines. More preferred are human lymphoma cell lines including, but not limited to, human B cell lines generated from patients with Burkitt's lymphoma.

Exemplary suitable cell lines are Ramos, Ramos sub-line 2G6, Burkitt's lymphoma cell lines BL2 and BL16, and BL16 sub-line CL-01. Such cell lines constitutively express the gene encoding activation-induced cytidine deaminase (AICD), or can be induced to express AICD, and, thereby, somatically mutate immunoglobulin genes. Denepoux et al., Immunity 6(1):35-46 (1997) and Sale et al., Immunity 9(6):859-69 (1998). The Ramos sub-line (2G6) also undergoes immunoglobulin isotype switching. Lederman et al., J. Immunol. 152(5):2163-71 (1994), and a sub-line of the BL16 Burkitt's lymphoma called CL-01 has been shown to undergo very efficient immunoglobulin isotype switching and immunoglobulin somatic mutation. Cerutti et al., J. Immunol. 160(5):2145-57 (1998).

In addition to Ramos, BL16, and CL-01, the present invention may also employ alternative cell lines that are capable of expressing immunoglobulin genes and, optionally, that undergo isotype switching and/or somatic mutation. Thus, methods of the present invention may be suitably employed in any number of immunoglobulin expressing cells and cell lines that are available in the art or that may be generated through routine experimentation.

### Reversion of V(D)J Recombined Immunoglobulin Genes in Immunoglobulin Expressing Cells to Germline-Like or Pro-B Cell-Like States

Immunoglobulin expressing cells, including B cells and B cell lines, are characterized by stages in development defined by the sequential rearrangement and expression of heavy and light chain immunoglobulin genes. The earliest B-lineage cells are commonly referred to as pro-B cells and are derived from pluripotent hematopoietic stem cells wherein the immunoglobulin genes are in the germline state. Rearrangement of heavy chain immunoglobulin regions takes place in pro-B cells, D to J joining at the early pro-B cell stage is followed by V to D joining at the late pro-B cell stage.

As part of the present invention, it was determined that a cell, such as an immunoglobulin expressing cell, comprising a rearrangement of its VDJ IgH regions and/or VJ IgL regions can be reverted to a more germline-like or pro-B cell-like state by introducing into the cell a chromosome or polynucleotide comprising one or more V, D, and/or J regions. As used herein, the terms “revert” and “reversion” refer to the process whereby a cell having rearranged V, D, and/or J regions is converted to a germline-like or pro-B cell-like state.

In a cell that is reverted to a germline-like state, one or more of the cell's rearranged V(D)J regions are replaced with unrearranged V, D, and/or J regions. As discussed in further detail below, a cell may be reverted to a germline-like state by introducing one or more chromosome, or substantial portion thereof, containing unrearranged V, D, and/or J regions. In such a case, one or more endogenous VDJ IgH rearrangement and/or one or more VJ IgL rearrangement may be replaced by homologous recombination between the introduced chromosome's unrearranged V, D, and/or J sequences and sequences comprising one or more endogenous V(D)J rearrangement. Alternatively, one or more endogenous VDJ IgH rearrangement and/or one or more VJ IgL rearrangement may be replaced with unrearranged V, D, and/or J sequences by spontaneous loss of an endogenous chromosome containing a V(D)J rearrangement and retention of one or more introduced chromosome, or substantial portion thereof.

In a cell that is reverted to a pro-B cell-like state, one or more of the cell's rearranged VDJ IgH regions is replaced with one or more DJ rearrangements by homologous recombination between sequences of an introduced polynucleotide comprising the DJ rearrangements and one or more endogenous rearranged VDJ IgH regions. Alternatively, or additionally, a cell may be reverted to a pro-B cell-like state by replacing one or more of the cell's rearranged VJ IgL (i.e. Igκ and/or Igλ) regions with one or more unrearranged J regions by homologous recombination between sequences of an introduced polynucleotide comprising unrearranged J regions and one or more endogenous rearranged VJ IgL regions. Thus, within the context of a pro-B cell-like state, the term “reversion” encompasses replacement of one or more of a cell's rearranged IgH and/or IgL regions.

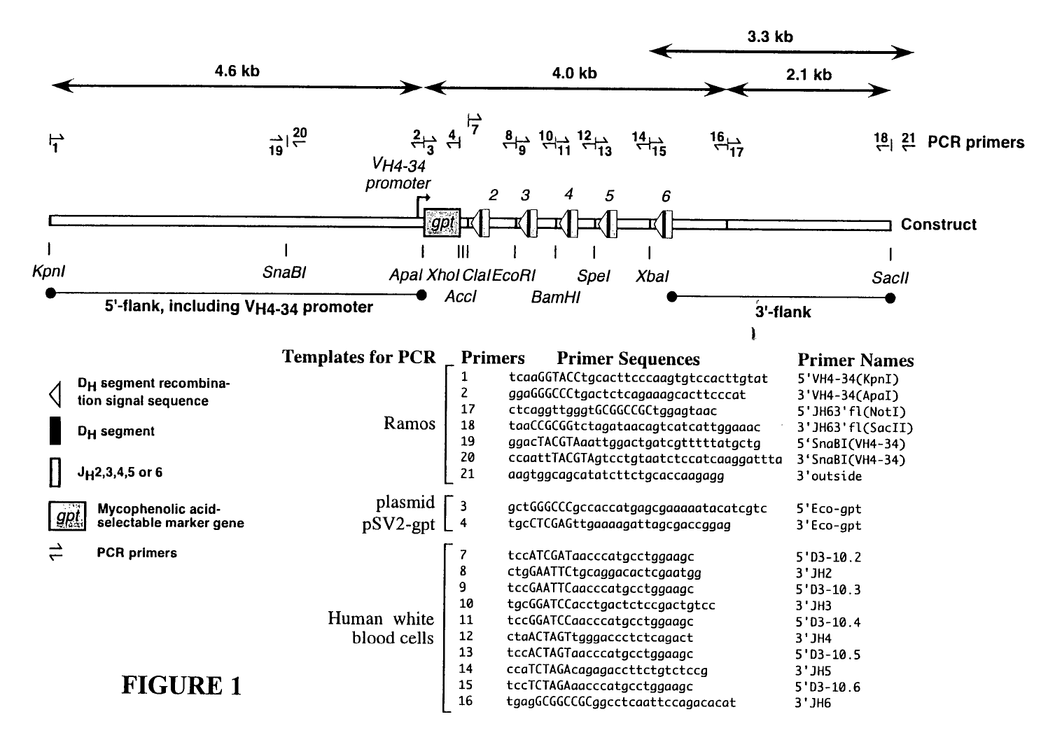

In those embodiments of the present invention wherein cells containing V(D)J rearranged immunoglobulin genes are reverted to a pro-B cell-like state (i.e. D and J regions joined in the immunoglobulin heavy chain locus, but otherwise all immunoglobulin V and J regions separate; Hardy et al., J. Exp. Med. 173:1213-1225 (1991)), reversion may be achieved by introducing a vector, such as a plasmid vector, comprising a polynucleotide encoding fused DJ regions of an immunoglobulin heavy chain, or fragment thereof. Within certain methods, the introduced polynucleotide comprises two or more fused DJ regions. Other methods provide that the polynucleotide comprises at least three, four, or five fused DJ regions. Still further methods provide that the polynucleotide comprises six fused DJ regions. An exemplary polynucleotide comprising six fused DJ regions (pro-B IgH; PH017) is presented herein in FIG. 1. The nucleotide sequences of pro-B IgH (PH017) is presented herein as SEQ ID NO: 68.

Alternatively, cells containing V(D)J rearranged immunoglobulin genes may be reverted to a germline-like state by either (1) introducing a vector comprising a polynucleotide encoding V, D, and/or J regions of immunoglobulin heavy and/or light chains, or fragments thereof, wherein the V, D, and/or J regions are assembled in the vector in a germline-like state or (2) introducing a polynucleotide encoding V, D, and/or J regions of an immunoglobulin heavy and/or light chain, or fragment thereof as a chromosome, or substantial portion thereof, that encodes a complete germline heavy or light chain antibody repertoire or substantial portion thereof. Regardless of the methodology chosen to achieve a germline-like state, the genetic modification of an immunoglobulin expressing cell or cell line results i n the replacement o f t he V (D)J-rearranged antibody genes with arrangements more closely resembling those seen in the germline (i.e. all V, D and J immunoglobulin heavy and light chain regions separate one from the other).

(a) Revertiniz Immunoglobulin Expressing Cells to a Pro-B Cell-Like State

Thus, the present invention provides vectors, including plasmid vectors, useful in reverting cell lines to a pro-B cell-like state or to a germline-like state, the vectors comprising one or more antibody regions including V regions, D regions, and/or J regions as well as any combination thereof. More preferred vectors comprise D and/or J regions. Still more preferred are vectors for reverting cell lines to a pro-B cell-like state wherein such vectors comprise fused DJ regions that approximate a rearranged form. Each of the vectors disclosed herein facilitate replacement of the endogenous V(D)J rearrangement by introducing V, D, and/or J regions by homologous recombination. An exemplary plasmid vector for introducing fused DJ_{H} regions by homologous recombination is presented herein in FIG. 1. The nucleotide sequences of pro-B IgH (PH017) is presented herein as SEQ ID NO: 68.

To achieve fused DJ_{H} regions, one or more of the 27 endogenous D_{H} regions may be joined to one or more of the 6 endogenous J_{H} regions. D_{H} and J_{H} regions that have already undergone D-J rearrangement in vivo may, for example, be PCR amplified from genomic DNA isolated from human peripheral blood lymphocytes, as exemplified herein. Alternatively, individual D and J regions may be PCR amplified independently and conventional recombinant DNA methodology utilized to create fused DJ regions. Vectors may be constructed by incorporating one or more DJ_{H} region operably linked to a selectable marker, such as, for example, the E. coli gpt (Eco-gpt) gene (disclosed herein in SEQ ID NO: 27), to permit selection of effective recombination into the genome of the immunoglobulin expressing cell, for example, by conferring upon the reverted cell resistance to mycophenolic acid in the presence of added xanthine and hypoxanthine.

Vectors of the present invention further comprise a 5′ flanking region and a 3′ flanking region for facilitating homologous recombination of the antibody regions into a cell having a rearranged immunoglobulin gene wherein the 5′ flanking region is operably linked 5′ to the 5′-most fused DJ region and wherein the 3′ flanking region is operably linked 3′ to the 3′-most fused region.

The precise sequence of the 5′ and 3′ flanking region will vary depending upon the immunoglobulin expressing cell utilized but, in all cases, are designed to enable the reversion of the immunoglobulin expressing cell from a V(D)J or a VJ recombined state. V(D)J and VJ rearrangements in immunoglobulin expressing cells may be cloned as inserts from genomic DNA libraries or by PCR amplification directly from genomic DNA and subjected to DNA sequencing methodology to determine the nucleotide sequences that flank the unique V(D)J or VJ rearrangements. 5′ flanking regions comprise genomic sequence that is 5′ to the endogenous V(D)J or VJ rearrangement and 3′ flanking regions comprise genomic sequence that is 3′ to the endogenous V(D)J or VJ rearrangement. Within certain embodiments, the combination of 5′ and 3′ flanking regions permits the homologous recombination of fused DJ_{H} regions 3′ to unrearranged endogenous V regions and 5′ to the intronic enhancer upstream of immunoglobulin light and/or heavy chain constant (C) region exons. It will be understood, however, that the precise sequence of the 5′ and 3′ flanking regions will depend upon the sequences flanking the endogenous V, D, and/or J regions. Flanking regions suitable for constructing the vectors disclosed herein are generally at least 250 bp, most commonly at least 500, 1,000, 2,000, 5,000, and/or 10,000 bp.

Successful reversion of immunoglobulin expressing cells may be selected for, as indicated above, by utilizing a selectable marker, such as, but not limited to the bacterial neoR and gpt genes or fragments or derivatives thereof. In the specific case of immunoglobulin expressing cells, such as Burkitt's lymphoma cells, that normally express immunoglobulins on their surface in the context of a B cell receptor complex, successful reversion of the immunolobulin genes to a pro-B cell-like state may be monitored by detecting the stable loss of expression of immunoglobulin on the surface of reverted cells using such standard cell biology techniques as fluorescence-activated cell sorting (FACS) analysis.

(b) Reverting Immunoglobulin Expressing Cells to a Germline-Like State

An alternative approach to introducing the antibody germline into an immunoglobulin expressing cell or cell line, such as, for example the Ramos B cell-line, is through the introduction of a chromosome, or substantial portion thereof, comprising a sequence encoding V, D, and/or J regions of an immunoglobulin heavy and/or light chain, or fragment thereof wherein the chromosome encodes a germline heavy or light chain antibody repertoire. As used herein, a “substantial portion” of a chromosome refers to a portion of a chromosome that comprises each of the essential elements of that chromosome. Thus, for example, a “substantial portion” of a chromosome will contain a centromere, two (2) telomeres, and one or more origins of replication. Generally, a “substantial portion” of a chromosome encompasses a linear, generally megabase long, DNA segment that stably replicates in a vertebrate cell independent of integration and/or joining onto an endogenous chromosome. As used herein within the context of a chromosome, the phrase “substantial portion” is meant to include engineered “mini chromosomes” and “artificial chromosomes” as these terms are understood by those of skill in the art.

An exemplary source of chromosomes that may be suitably introduced into immunoglobulin expressing cells according to the methods of the present invention include panels of mouse cell lines carrying a single intact human chromosome. Cuthbert et al., Cytogenetics & Cell Genetics 71(1):68-76 (1995); Ning et al., Cytogenetics & Cell Genetics 60(1):79-80 (1992) and Ning et al., Genomics 16(3):758-760 (1993). Each cell line in the Cuthbert panel (named Hytk-1 through to Hytk-22) carries a single human autosomal chromosome tagged with a synthetic fusion gene (Hytk) that confers resistance to the antibiotic hygromycin (via the hyg protein) and susceptibility to the antiviral ganciclovir (via the tk protein). Each cell line in the Ning panel carries a single human autosomal chromosome tagged with the neo^{R} gene that confers resistance to the antibiotic G418. The relevant lines are named A9+2 and A9+22. The human chromosomes in these panels are derived from fibroblasts, so the immunoglobulin genes on human chromosomes 2, 14 and 22 are in a completely unrearranged germline configuration.

Human chromosomes, such as human chromosome 14 carrying the entire immunoglobulin heavy chain locus from A9-Hytk14 cells, may be transferred to immunoglobulin expressing cells, including immunoglobulin-positive Burkitt's lymphoma cells, using microcell-mediated chromosome transfer (MMCT). Introduction of the polynucleotide encoding immunoglobulin heavy chain V, D, and J regions may be selected for by screening immunoglobulin expressing cells for clones that are positive for hygromycin resistance, i.e. that have acquired the Hytk gene. Clones that lose the Ramos canonical VDJ_{H}-rearrangement and keep the germline IgH gene segments introduced by MMCT may be subsequently selected by identifying cells that are stably surface immunoglobulin negative and also negative in a PCR-based screen that detects the Ramos canonical VDJ_{H}-rearrangement. Alternatively, clones that spontaneously undergo homologous recombination involving the introduced chromosome 14 and the endogenous chromosome 14 carrying the V(D)J_{H} rearrangement may be subsequently selected by identifying cells that are stably surface immunoglobulin negative and negative in a PCR-based screen that detects the Ramos canonical VDJ_{H}-rearrangement, and are also ganciclovir-resistant, (that is, possibly reverted by a recombination at the immunoglobulin heavy chain locus that has caused the loss of the Hytk gene).

This same methodology with minor variations may also be employed to introduce a chromosome, or substantial portion thereof, comprising a sequence encoding V and J regions of immunoglobulin light chains, or fragments thereof as chromosome 2 and 22, or a substantial portion thereof, that encode the complete germline kappa and lambda light chain antibody repertoires, respectively, or substantial portions thereof. “Ig germline” light chain chromosomes would be most simply introduced into IgH-reverted lymphoma cells by fusion between IgH-reverted lymphomas and microcells derived from A9+2 or A9+22, because the antibiotics G418 and hygromycin could be used together to select lymphomas that were both IgH- and IgL-germline reverted. Alternatively, “Ig germline” light chain chromosomes could be introduced into IgH-reverted lymphoma cells by fusion between ganciclovir-resistant IgH-reverted lymphomas and microcells derived from A9-Hytk2 or A9-Hytk22 cells, with repetition of the use of hygromycin selection.

The introduction of chromosomes, or substantial portions thereof, comprising sequences encoding V, D and/or J regions of immunoglobulin light chains, or fragments thereof has at least two advantages over the use of vectors to achieve a germline-like state in immunoglobulin expressing cells. First, the direct transfer of chromosomes obviates the construction of vectors. Second, the introduction of whole chromosomes means that the resulting cell lines have the potential for recombining the complete complement of immunoglobulin V, D and/or J regions thereby maximizing the potential diversity of immunoglobulin cell libraries produced by the methods of the present invention.

Chromosomes may be introduced into a cell by a methodology including, but not limited to, microcell-mediated chromosome transfer (MMCT), T cell-fusion, micro-injection, and/or yeast protoplast fusion. Within certain aspects, lymphoma cell lines containing V(D)J rearranged immunoglobulin genes are reverted to a germline-like configuration by fusing a B cell line with precursor B cells isolated from human bone marrow or cord blood. Other aspects provide that lymphoma cell lines may be reverted to a germline-like configuration by fusing a B cell line with T cells isolated from human blood. Still further aspects provide that lymphoma cell lines are reverted to a germline-like configuration by fusing B cell lines with rodent/human somatic cell hybrids carrying single or mutiple human chromosomes.

### Promoting Recombination of V, D, and/or J Regions by Introducing a Polynucleotide Encoding a Recombination-facilitating Protein

Recombination between V, D, and/or J regions in a reverted cell may be achieved by introducing into the reverted cell a polynucleotide encoding one or more “recombination-facilitating proteins,” such as the proteins RAG-1, RAG-2, and TdT that collectively constitute a “V(D)J recombinase.” In combination with housekeeping DNA repair pathways, the two RAG proteins are sufficient to carry out immunoglobulin V(D)J recombination in any cell while the TdT protein is required for the insertion of random “N” nucleotides into the V(D)J junctions. Oettinger et al., Science 248:1517-1523 (1990) and Komori et al., Science 261:1171-1175 (1993).

Recombination-facilitating proteins induce, inter alia, rearrangement of immunoglobulin-encoding genes, preferably in a random manner that mimics the development of B cells in the mammalian or more preferably human body. As a result, a library of immunoglobulin expressing cells is generated that, instead of making one unique immunoglobulin, produce an array of different immunoglobulins and more particularly an array of different human monoclonal immunoglobulins.

Accordingly, in a preferred embodiment, there is provided a method for producing a library of human immunoglobulin-producing cells, the method comprising expressing one or more polynucleotides encoding a recombination-facilitating protein for a time and under conditions sufficient to induce rearrangement of genes encoding immunoglobulins in said reverted lymphoma cells and screening for cells that produce immunoglobulins.

As used herein, the term “recombination-facilitating protein” refers to those proteins, including functional derivatives, fragments, portions, mutants, variants and mimetics thereof, that are effective in facilitating recombination between immunoglobulin V, D, and/or J regions. Recombination-facilitating proteins, or functional fragments, derivatives or variants thereof, which are suitable in any of the methods disclosed herein may be selected from the group consisting of RAG-1 and RAG-2. Derivatives of polynucleotides encoding recombination-facilitating proteins include, but are not limited to, insertions, deletions, and substitutions of nucleotides within the polynucleotide coding region. Nucleotide insertional derivatives include 5′ and/or 3′ terminal fusions as well as intrasequence insertions of single or multiple nucleotides.

RAG-1 and RAG-2 proteins may be from any species including primates, livestock animals, laboratory test animals and companion animals as well as functionally similar molecules from avian, reptilian, amphibian or aquatic animals. More preferred are RAG-1 and RAG-2 proteins from humans such as those provided herein as SEQ ID NOs: 2 and 4, respectively.

Recombination-facilitating proteins, or functional fragments, derivatives or variants thereof, may be expressed transiently for a time and under conditions sufficient to achieve recombination in any of the reverted cells recited herein. Within other embodiments, the recombination-facilitating proteins may be expressed constitutively and expression of these proteins may be under the control of an inducible transcriptional promoter. cDNAs encoding the human proteins RAG-1, RAG-2, and TdT are presented herein as SEQ ID NOs: 1, 3, and 5, respectively. These sequences may be cloned singularly or in tandem into a vector, such as a plasmid vector, that drives expression of the introduced cDNAs in immunoglobulin expressing cells, such as Burkitt's lymphoma cells. Vectors carrying cDNAs encoding RAG-1, RAG-2, and TdT, or functional fragments, derivatives, and variants thereof, may be co-introduced into the immunoglobulin gene-reverted cell or cell-lines.

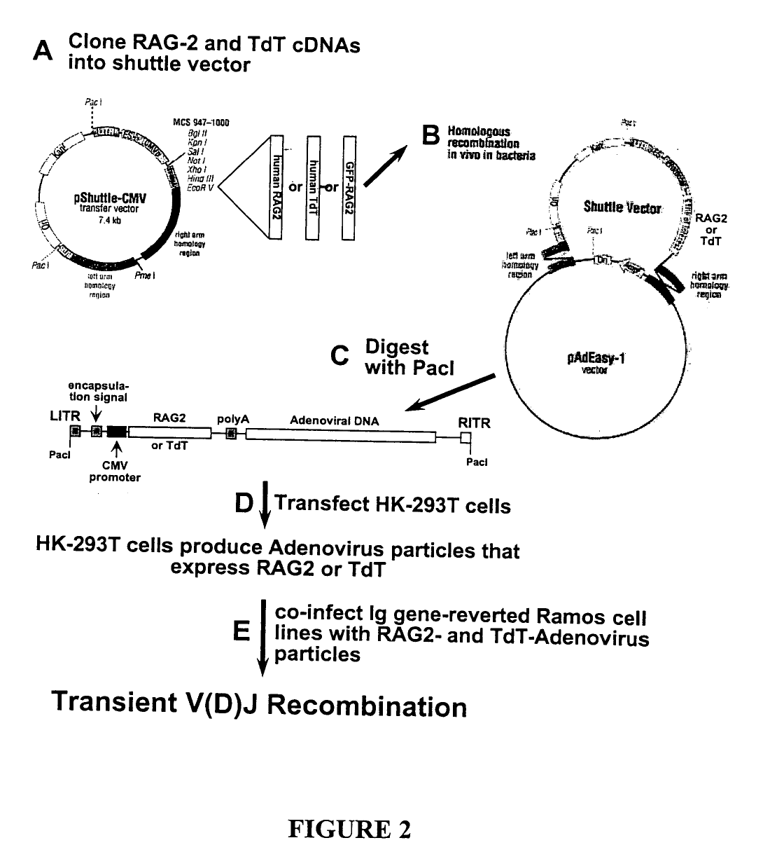

As exemplified herein, vectors comprising polynucleotides encoding recombination-facilitating proteins are Adenoviral vectors such as pAdEasy-1 (Stratagene, La Jolla, Calif., USA) exemplified in FIG. 2, packaged into recombinant Adenoviral particles after electroporation into packaging cell lines such as HK293T, and finally introduced into reverted B cells by adenoviral transduction. Genes carried in Adenoviral vectors transduced into Burkitt's lymphomas should be expressed in greater than 10% of the cells in culture, more preferably, in greater than 20%, 30%, 50%, or 70% of the cells in culture for a period of hours to days. Most preferably, the Adenoviral vectors are expressed in greater than 90% of the cells in culture for a period of hours to days. The adenoviral DNA (and hence RAG-1, RAG-2 and/or TdT expression) is lost as the cells divide. Successful expression of a functional recombination-facilitating protein is indicated by the appearance of immunoglobulin-positive cells detected by FACS in the transduced populations (that were originally stably immunoglobulin-negative).

It will be appreciated that alternative vector systems may be employed in the methods of the present invention to achieve expression of one or more recombination-facilitating protein. Vectors, including alternative viral vectors and plasmid vectors, may be fashioned to express, for example, Rag-1 and/or Rag-2 or functional fragments, derivatives, and variants thereof. Such vectors may be constructed and introduced into cells by conventional molecular and cell biology methodologies that are readily available in the art and that may be performed through routine experimentation.

Immunoglobulin-positive cells may be purified using any methodology commonly available in the art including, but not limited to, magnetic beads conjugated to anti-immunoglobulins, preferably anti-human immunoglobulins.

Multiple introductions of vectors comprising recombination-facilitating protein coding regions may be carried out to ensure as much diversity as possible in the immunoglobulin V(D)J recombinations produced. Growing the surface immunoglobulin-positive cells in culture will expand these libraries. The resulting libraries can be frozen down for long-term storage in unexpanded and expanded forms.

### Screening for Antigen-Specific Immunoglobulins

Within certain embodiments, methods of the present invention may further provide a step of screening V, D, and/or J region rearranged cells for immunoglobulins having a desired antigen binding specificity and/or affinity. As used herein, the term “antigen” broadly encompasses all those substances, molecules, proteins, nucleic acids, lipids and/or carbohydrates to which an immunoglobulin specifically binds and/or interacts.

Immunoglobulins may be screened for preferred antigen binding specificity and/or affinity by any of the methodologies that are currently available in the art. F or example, conventional cell panning, Western blotting and ELISA procedures may be employed to accomplish the step of screening for immunoglobulins having a particular specificity. A wide range of suitable immunoassay techniques is available as can be seen by reference to U.S. Pat. Nos. 4,016,043, 4,424,279, and 4,018,653, each of which is incorporated herein by reference.

In one type of assay, an unlabelled anti-immunoglobulin is immobilized on a solid support and the immunoglobulin-containing sample to be tested is brought into contact with the immobilized anti-immunoglobulin. After a suitable period of time sufficient to allow formation of a first complex, a target antigen labeled with a reporter molecule capable of producing a detectable signal is then added and incubated, allowing time sufficient for the formation of a second complex of immobilized anti-immunoglobulin/immunoglobulin sample/test antigen. Uncomplexed material is washed away, and the presence of the antigen is determined by observation of a signal produced by the reporter molecule. The results may either be qualitative, by simple observation of the visible signal, or may be quantified by comparison with a control sample containing known amounts of antigen or antibody. Variations of this type of assay include a simultaneous assay, in which both sample and labeled antigen are added simultaneously to the bound antibody.

In a second type of assay, an antigen for which an immunoglobulin is sought is bound to a solid support. The binding processes are well known in the art and generally consist of cross-linking, covalently binding or physically adsorbing the antigen to the solid support. The polymer-antigen complex is washed in preparation for the test sample. An aliquot of the sample to be tested is then added to the solid phase complex and incubated for a period of time sufficient (e.g., 2-40 minutes or overnight if more convenient) and under suitable conditions (e.g., from about room temperature to about 38° C., such as 25° C.) to allow binding of immunoglobulin to the antigen. Following the incubation period, the solid support is washed and dried and incubated with an immunoglobulin to which a reporter molecule may be attached thereby permitting the detection of the binding of the second immunoglobulin to the test immunoglobulin complexed to the immobilized antigen.

Suitable solid supports include glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay.

An alternative assay system involves immobilizing the target immunoglobulin and exposing the immobilized target immunoglobulin to an antigen that may or may not be labeled with a reporter molecule. As used herein, the term “reporter molecule” refers to a molecule that, by its chemical, biochemical, and/or physical nature, provides an analytically identifiable signal that allows the screening for immunoglobulins complexed with antigens or with second immunoglobulins. Detection may be either qualitative or quantitative. The most commonly used reporter molecules employed in assays of the type disclose herein are enzymes, fluorophores, radioisotopes, and/or chemiluminescent molecules. In one particularly useful assay system, cultures of cell libraries expressing immunoglobulins in the context of B cell receptors, on the surface of the cells, may be incubated with antigen coupled to a label, such as biotin, or carrying a recombinant epitope, such as the FLAG epitope. Castrucci et al. J. Virol. 66:4647-4653 (1992). After a suitable incubation time, the labeled antigen is washed away by pelleting the cells two or three times from suspension in ice-cold medium.

The cells may be further incubated in medium containing a suspension of beads conjugated to a reagent that will specifically bind the label attached to the antigen (e.g., streptavidin or avidin for the biotin label, or anti-FLAG antibody for the FLAG epitope). Cells that bind to the beads as an indirect consequence of binding the antigen of interest are separated from the remaining cells by appropriate means, and then returned to tissue culture to proliferate. Repetition of this process using increasingly limiting amounts of antigen results in enrichment for cells that bind to the antigen specifically and/or with high affinity. Cells that bind directly to the beads independently of prior binding to antigen may be removed by incubation with beads in the absence of antigen. Individual antigen-binding clones may then be purified, such as for example by fluorescence-activated cell sorting (FACS), after labeling the cells with antigen conjugated directly or indirectly to a suitable fluorochrome such as fluorescein.

In the case of an enzyme immunoassay (EIA), an enzyme is conjugated to the detection immunoglobulin, generally by means of glutaraldehyde or periodate. As will be readily recognized, however, a wide variety of different conjugation techniques exist, which are readily available to the skilled artisan. Commonly used enzymes include horseradish peroxidase, glucose oxidase, P-galactosidase and alkaline phosphatase. In general, the enzyme-labeled immunoglobulin is added to a potential complex between an antigen and an immunoglobulin, allowed to bind, and then washed to remove the excess reagent. A solution containing the appropriate substrate is then added to the complex of antigen/test-immunoglobulin/labeled-immunoglobulin. The substrate reacts with the enzyme linked to the second antibody, giving a qualitative visual signal, which may be further quantified, usually spectrophotometrically, to indicate the amount of immunoglobulin present in the sample.

Alternatively, fluorescent compounds, such as fluorescein and rhodamine, or fluorescent proteins such as phycoerythrin, may be chemically coupled to immunoglobulins without altering their binding capacity. When activated by illumination with light of a particular wavelength, the fluorochrome-labeled immunoglobulin absorbs the light energy, inducing a state of excitability in the molecule, followed by emission of the light at a characteristic color visually detectable with a light microscope or other optical instruments. As in the EIA, the fluorescent labeled immunoglobulin is allowed to bind to the antigen-antibody complex. After removing unbound reagent, the remaining tertiary complex is exposed to light of the appropriate wavelength. The fluorescence observed indicates the presence of the bound antibody of interest. Immunofluorescence and EIA techniques are both well established in the art. It will be understood that other reporter molecules, such as radioisotopes, and chemiluminescent and/or bioluminescent molecules, may also be suitably employed in the screening methods disclosed herein.

### Altering the Affinity of Antibodies Expressed by a Single B Cell Clone

Further provided herein are methodologies for altering the antigen binding affinity and/or specificity of an immunoglobulin produced by the methods of the present invention. Once immunoglobulin producing cells are identified, the cells may subsequently be subjected to one or more mutagenesis methodologies in an effort to, for example, increase efficiency of immunoglobulin production and/or increase efficiency of binding to, and/or neutralizing, target molecules of interest.

The affinity and/or specificity of individual immunoglobulin expressing cells may be altered by inducing mutations into the immunoglobulin coding region. Mutagenesis may be achieved by a variety of methodologies currently available in the art such as, for example, chemical mutagenesis and UV irradiation.

Preferred methodologies utilize the cell's ability to randomly generate somatic mutations within the gene encoding the immunoglobulin. As indicated above, Burkitt's lymphoma cells have an innate capacity to mutate their antibody genes. Such an ability to mutate these genes may be exploited to generate immunoglobulins exhibiting one or more of these desired properties. Somatic mutation may, for example, be induced in Burkitt's lymphomas by cross-linking the surface Ig antigen receptor in the presence of activated human T cells. Denepoux et al., Immunity 6(1):35-46 (1997) and Zan et al., J. Immunol. 162(6):3437-47 (1999).

In those immunoglobulin expressing cells that do not undergo constitutive or inducible somatic mutations, an alternative methodology may be employed wherein the polynucleotide encoding the activation-induced cytidine deaminase (AICD) may be introduced into and expressed exogenously within the cell. AICD is required for somatic hypermutation (SHM) and isotype-switch recombination (CSR) of immunoglobulin (Ig) genes, both of which are associated with DNA double-strand breaks (DSBs). Without being limited to any specific theory of operation, it is thought that because AICD is capable of deaminating deoxy-cytidine (dC) to deoxy-uracil (dU), AICD can induce DNA transitions directly by deaminating deoxy-cytidine residues in immunoglobulin genes and to subsequently induce transversions via a dU-DNA glycosylase-mediated base excision repair pathway (‘DNA-substrate model’). Petersen-Mahrt et al., Nature 418(6893):99-103 (2002); and Di Noia et al., Nature 419(6902):43-48 (2002). Preferred methods comprise the step of introducing into the cell and/or library of cells a polynucleotide encoding human activation-induced cytidine deaminase (AICD) provided herein as SEQ ID NO: 38.

Further aspects of the present invention provide that the antigen specificity and/or affinity of immunoglobulins generated by any of the methods presented herein may be altered by introducing into the cells and/or libraries of cells a polynucleotide encoding Terminal Deoxynucleotidyltransferase (TdT) or a functional fragment, derivative or variant thereof. Most preferred is human TdT as disclosed herein in SEQ ID NO: 6. Preferably, the polynucleotide encoding TdT is introduced into the cells and/or library of cells coincident with the introduction of the polynucleotide(s) encoding the recombination-facilitating protein.

### Switching Antibody Isotype

Depending on the particular application contemplated, it may be desired to switch the isotype of immunoglobulin(s) generated by the methods of the present invention. Immunoglobulins generated by the methods disclosed herein are principally of the IgM isotype. Thus, further aspects of the present invention provide that cells expressing immunoglobulins and fragments thereof, generated by any of the methods presented herein may be further treated with an agonist, or combination of agonists, to induce switching from a first immunoglobulin isotype to a second immunoglobulin isotype. Within these aspects, the first immunoglobulin isotype may be selected from the group consisting of IgM, IgD, IgG1, IgG2, IgG3, IgG4, IgE, IgA1 and IgA2; the second immunoglobulin isotype may be selected from the group consisting of IgD, IgG1, IgG2, IgG3, IgG4, IgE, IgA1 and IgA2.

In one embodiment, the most effective isotype of immunoglobulins for a particular application are selected by exposing antigen-specific immunoglobulin expressing cells in tissue culture to combinations of agonists known to induce “switching” in antibody genes. As used herein, the term “isotype switching” refers to the recombination event whereby the Switch (S) region immediately 3′ to the active heavy chain V region exon undergoes somatic recombination with an S region associated with a 3′ constant region gene. Every immunoglobulin-expressing cell begins by expressing IgM. The same assembled V region may be expressed in IgG, IgA, or IgE immunoglobulins by a recombination event stimulated by one or more agonists, for example, cytokines released by T cells.

By way of example, not limitation, treatment of Burkitt's lymphoma cells with appropriate cytokines and/or mitogens induces switching away from the starting isotype (IgM) to other predictable Ig isotypes. Ledermnan et al., J. Immunol. 152(5):2163-71 (1994) and Cerutti et al., J. Immunol. 160(5):2145-57 (1998). Prolonged stimulation can also cause differentiation into plasmacyte-like cells that secrete large amounts of antibody. Cerutti, et al., supra. For example, CD40 ligand in conjunction with IL4 and/or IL10 may be employed to induce switching from IgM to IgG.

Suitable agonists for inducing isotype switching include, but are not limited to, (1) ligands for CD40 such as CD154, anti-CD40 or fresh T cells activated with, for instance, phytohaemoglutenin; (2) ligands for the B cell receptor such as anti-Ig or anti-Ig coupled to dextran; (3) B cell mitogens such as purified protein derivative from Mycobacterium species (PPD), bacterial DNA, or synthetic oligonucleotides containing unmethylated CpG dinucleotides; (4) cytokines such as IL2, IL4, IL5, IL6, IL10, IL13, TGFβ or IFNγ; (5) anti-CD19; and/or (6) anti-CD21. Cells expressing immunoglobulins may be exposed to one or more of these agonists alone or in combination.

### Utility of Immunoglobulins of the Present Invention

Immunoglobulins of the present invention are useful as diagnostic and therapeutic agents. With respect to their use as diagnostic agents, antigenic molecules may be detected using the immunoglobulin employing assays such as described herein. Useful target molecules for diagnostic purposes include microoganisms and eukaryotic cells or components thereof. Such components include receptors, flagella, pilli, polysaccharide, proteins, phospholipids, enzymes, nucleic acids and ribozymes amongst others. Examples of eukaryotic cells include yeast, fungi, animal cells, mammalian cells, human cells, parasitic cells, cancer cells and normal cells.

The immunoglobulins may also be employed as therapeutic agents including active ingredients in a pharmaceutical composition. Therefore, in further aspects of the present invention, the immunoglobulins described herein may be used to stimulate an immune response against cancer. Within certain embodiments, immunotherapy may be active immunotherapy, in which treatment relies on the in vivo stimulation of the endogenous host immune system to react against tumors.

The following Examples are offered by way of illustration not limitation.

### EXAMPLES

### Example 1

### Reversion of Immunoglobulin Expressing Lyphoma Cell Lines to a “Pro-B Cell-Like” Arrangement by Homologous Recombination

This Example demonstrates the construction of vectors and methodology suitable for reverting a V(D)J rearranged immunoglobulin expressing cell line to a pro-B cell-like state.

Burkitt's lymphoma cell lines BL2 and Ramos can mutate their antibody genes. Denepoux et al., Immunity 6:35-46 (1997) and Sale et al., Immunity 9:859-869 (1998). A Ramos sub-line (2G6.4C10) is also known to undergo Ig isotype switching and a sub-line of the BL16 Burkitt's lymphoma (CL-01) has been shown to undergo both efficient Ig isotype switching and Ig somatic mutation. Lederman et al., J. Immunol. 152:2163-2171 (1994); Zan et al., J. Immunol. 162:3437-3447 (1999); and Cerutti et al., J. Immunol. 160:2145-2157 (1998). These lines are therefore all useful as starting immunoglobulin expressing lymphoma cell lines for the production of reverted immunoglobulin expressing lymphoma lines.

(a) Cloning of Immunoglobulin Heavy Chain D to J Rearrangements Lacking a V Region

Immunoglobulin heavy chain D to J rearrangements utilizing several or all six of the human J regions, but lacking a V region are cloned from human peripheral blood lymphocytes (PBLs) by PCR as follows. Human PBLs are prepared from a healthy donor using standards means. Colligan et al., “Current Protocols in Immunology” (John Wiley & Sons Inc, 1999). A sense oligodeoxynucleotide complementary to sequences upstream (5′) of the recombination signal sequence (RSS) belonging to human D region D3-10 (a commonly used D region) is paired with an antisense oligodeoxynucleotide complementary to sequences downstream (3′) of the RSS of each one of the six human J gene segments in 6 separate PCR reactions using DNA extracted from human PBLs as template. Corbett et al., J. Mol. Biol. 270:587-597 (1997). The sequences on which the primers (primers 5-15 correspond to SEQ ID NOs: 15-26) are based are in Genbank accessions AB019437-AB019441 (presented herein as SEQ ID NOs: 30-34). The DJ1, DJ2, DJ3, DJ4, DJ5, and DJ6 PCR products are cloned in an array immediately 3′ to the VH4-34 promoter and gpt selectable marker sequences. FIG. 1.

(b) Assembly of the Ramos Immunoglobulin V(D)J Replacement Vector

A region of approximately 4.6 kb immediately flanking the 5′ end of the Ramos canonical V(D)J rearrangement and including the promoter attached to the V_{H}4-34 gene segment is amplified by PCR using primers “5′VH4-34(KpnI)” and “3′VH4-34(ApaI)” (primers 1 and 2 in FIG. 1) and cloned immediately 5′ to the gpt selectable marker sequence, and a region of 2.1 kb flanking the 3′ end of the Ramos VDJ rearrangement is amplified by PCR using primers “5′JH63′fl(NotI)” and “3′JH63′fl(SacII)” (primers 17 and 18 in FIG. 1) and cloned immediately 3′ to the cluster of fused DJ regions to give the V(D)J-replacement construct disclosed in FIG. 1.

(c) Introduction of the Immunoglobulin VDJ_{H} Replacement Vector into Ramos

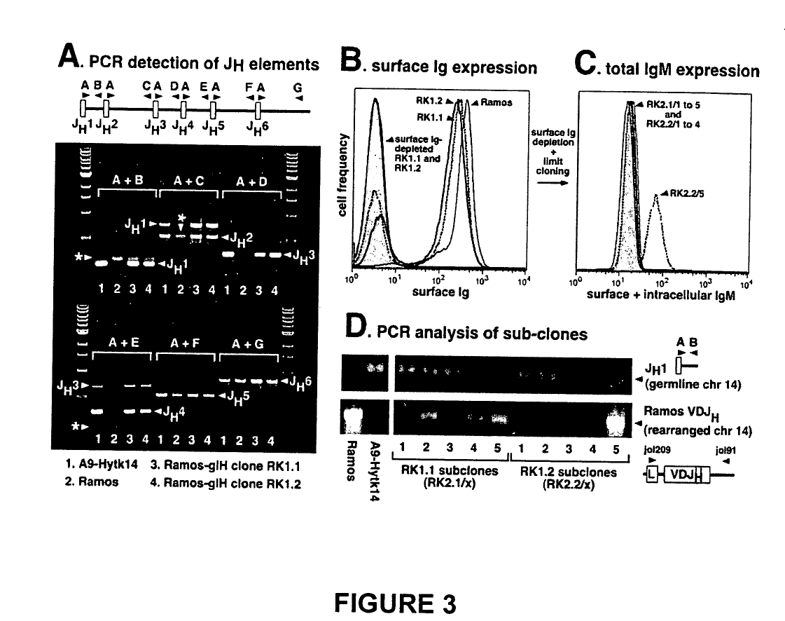

Ramos cells that are positive for surface Ig expression are selected by fluorescence-activated cell sorting (FACS) after staining with fluorescent anti-IgM antibodies. The vector described above and depicted in FIG. 1 is linearized and ˜20 μg is introduced by electroporation into these Ramos.2G6.4C10 cells (using 2×10⁷ cells, a 4 mM electrode gap, 0.25 kV, and 960 μF). After 24 hours in complete medium (RPMI containing 10% v/v fetal calf serum and 50 μM 2-mercaptoethanol), to allow the cells to recover from the shock of electroporation, the medium is supplemented with mycophenolic acid (2.5 μg/ml), xanthine and hypoxanthine. About 10-20 days after electroporation, clones that may have undergone homologous recombination with the introduced DNA are selected by the following two criteria: (A) resistance to mycophenolic acid which is conferred by expression of the gpt gene as a result of integration near enhancers functional in Ramos cells (e.g., the immunoglobulin heavy chain enhancers), and (B) stable loss of surface immunoglobulin expression (determined by FACS), possibly as a result of replacement of the expressed immunoglobulin heavy chain rearrangement with the VH4-34/gpt/DJ cassette. Homologous recombination is confirmed by Southern blot analysis and PCR-screening of mycophenolic acid-resistant IgM^{-ve} clones using (a) primer “5′D3-10.6” with primer “3′outside” (primers 15 and 21 in FIG. 1) and (b) primer jo1209 with primer jo191 (priming sites illustrated in FIG. 3D). DNA from homologously recombined clones produces a 3.3 kb PCR product when amplified with primers “5′D3-10.6” and “3′outside”, and fails to produce a product when amplified with primers jo1209 and jo191. Homologously recombined clones are called “Ramos.proB” cells.

It is not necessary to revert the Igλ locus to germline- or pro-B cell like arrangement in Ramos because functional Vλ segments and unrearranged Jλ gene segments are still present in both the expressed and non-expressed Ramos Igλ alleles. See, Sale et al., Immunity 9:859-869 (1998) and Corbett et al., J. Mol. Biol. 270:587-597 (1997).

### Example 2

### Reversion of Lymphoma Cell Lines to a “Germline-Like” Arrangement by Chromosome Replacement

This Example demonstrates the introduction of whole human chromosomes to achieve reversion of a V(D)J rearranged, immunoglobulin expressing cell line to a germline-like state.

The immunoglobulin heavy chain locus in human cells is on chromosome 14. Many Burkitt's lymphoma cell lines, including Ramos, carry one normal chromosome 14 from which the expressed immunoglobulin heavy chain is produced, and one abnormal chromosome 14 carrying a translocation to chromosome 8. The nature of the translocation in Ramos is such that the translocated chromosome 14 cannot express functional immunoglobulin heavy chain protein. Wiman et al., Proc. Nat. Acad. Sci. USA 81:6798-6802 (1984).

The normal chromosome 14 in Ramos, which carries the immunoglobulin heavy chain VDJ rearrangement, and one of the copies of chromosome 2, which carries the immunoglobulin kappa light chain locus, are replaced by chromosomes carrying the immunoglobulin heavy and kappa light chain loci in germline form by microcell-mediated chromosome transfer (MMCT) as outlined below.

Mouse A9 cells carrying a single copy of human chromosome 2, 14 or 22 have previously been produced. These cell lines are called A9+2, A9-Hytk2, A9-Hytk14, A9-Hytk22 and A9+22, respectively. Cuthbert et al., Cytogenetics & Cell Genetics 71(1):68-76 (1995); Ning et al., Cytogenetics & Cell Genetics 60(1):79-80 (1992) and Ning et al., Genomics 16(3):758-760 (1993). The human chromosome in cell lines A9+2 and A9+22 (chromosomes 2 and 22, respectively) is tagged by a provirus carrying a neomycin resistance gene (neo^{R}), while the human chromosomes in A9-Hytk2, A9-Hytk14 and A9-Hytk22 are tagged by a provirus carrying a hygromycin phosphotransferase (hph):herpes simplex virus thymidine kinase (HSVtk) fusion gene (called Hytk) driven by a proviral LTR. Id.

Microcells are made from cell line A9-HyTK14 and fused to Ramos cells using published methods. e.g. Hunt, Anal. Biochem. 238:107-116 (1996). After 48 hour in complete medium (RPMI+10% v/v fetal calf serum+50 μM 2-mercaptoethanol) to allow the Ramos cells to recover from the shock of fusion, the medium is supplemented with hygromycin B to a concentration of 800 units/ml. About 10-20 days after fusion, colonies that may have undergone chromosome replacement are selected by the following two initial criteria: (A) resistance to hygromycin B antibiotic (800 units/ml) which is conferred by expression of the HyTK gene as a result of acquiring all or part of the tagged human chromosomes carried by the A9HyTK14 cells, and (B) loss of surface immunoglobulin expression (determined by FACS), possibly as a result of replacement of the original Ramos chromosome 14 that expressed an immunoglobulin chain gene rearrangement. Hygromycin-resistant colonies containing high numbers of surface Ig-^{-ve} cells are depleted of surface Ig^{+ve} cells using biotin-conjugated anti-IgM antibody and streptavidin-coated magnetic beads and cloned by limiting dilution.

Acquisition of an array of germline immunoglobulin heavy chain gene segments is confirmed in these clones by PCR with primers that detect IgH gene segments normally absent from Ramos cells (for instance gene segments J_{H}1 to J_{H}4, see FIG. 3A) or by restriction mapping (See Matsuda et al., Nat. Genet. 3:88-94 (1993)). Loss of the VDJ-rearranged immunoglobulin heavy chain locus is detected by PCR with primers that detect the Ramos canonical VDJ_{H}-rearrangement (also see FIG. 3A). Clones that have acquired germline IgH gene segments and that have loss the Ramos canonical VDJ_{H}-rearrangement are selected and called “Ramos.glH” cells, where “gl” indicates that these cells contain immunoglobulin regions in a germline-like state. Microcells are then made from cell line A9+2 using the same procedure as above and separately fused to Ramos and Ramos.glH cells. After 48 hour in complete medium, the medium is supplemented with G418 to a concentration of 3 mg/ml. G418-resistant colonies are then screened by PCR (using primers jo1266-5′-GAC TCT AGA GCA GCT CCA AAG ATG GCA TGC G-3′ (SEQ ID NO: 57) and jo1267-5′-GTT TGA ATT CCA CCT TGG TCC CTT GG-3′ (SEQ ID NO: 58)) for the Jκ1 gene segment which is normally absent from Ramos cells. Clones that acquired the Jκ1 gene segment are selected. Clones resistant to both G418 and hygromycin (derived from MMCT between A+2 and Ramos.ghL cells) are called Ramos.glκH cells, while clones resistant to G418 only (derived from MMCT between A+2 and Ramos cells) are called Ramos.glκ cells.

Microcells are then made from cell line A9+22 using the same procedure as above and separately fused to Ramos and Ramos.glH cells. After 48 hour in complete medium, the medium is supplemented with G418 to a concentration of 3 mg/ml. G418-resistant colonies are then screened by PCR (using primers kam8-5′-CCT GCT CTG GAG ATA AAT TGG G-3′ (SEQ ID NO: 59) and kam9-5′-CTG CTG TCC CAC GCC TGA CA-3′ (SEQ ID NO: 60)) for the 3′ distal Vλ gene segment 3r (or DPL23) which is normally absent from Ramos cells. Clones that acquired Vλ gene segment 3r are selected. Clones resistant to both G418 and hygromycin (derived from MMCT between A+22 and Ramos.ghL cells) are called Ramos.glλH cells, while clones resistant to G418 only (derived from MMCT between A+22 and Ramos cells) are called Ramos.glλ cells.

As an alternative procedure for introducing germline IgL gene segments, Ramos cells or ganciclovir-resistant Ramos-glH cells (i.e. which have lost the Hytk gene by recombination) could be fused with microcells derived from A9-Hytk2 and A9-Hytk22 cells. Lymphoma cells that had taken up germline IgL gene segments would be detected by PCR screening (as above) of the hygromycin-resistant colonies generated.

(a) Reversion of a Ramos Cell Line to a “Germline-Like ” IgH Arrangement by Chromosome Replacement with Human Chromosome 14

Using MMCT, a human chromosome 14 carrying a germline IgH allele from A9-HytK-14 cells was transferred to Ramos. Several stable hygromycin resistant cell lines (designated RK1.1 to RKB1.9) were produced and shown by flow cytometry to be CD19-positive (data not shown) and to contain a mixture of surface Ig-positive and -negative cells. FIG. 3B. PCR amplification from genomic DNA was used to confirm that these cell lines carried J_{H} gene segments normally absent from Ramos cells. FIG. 3A. In order to select sub-lines that had lost the VDJ_{H}-rearrangement originally present in Ramos, cells from two of these lines were depleted of surface Ig-positive cells using magnetic beads then cloned by limiting dilution. FIGS. 3B and 3C. Some of these sublines (designated RK2.1/1, RK2.2/1, RK2.2/2, RK2.2/3 and RK2.2/4) expressed a permanent IgM^{-ve} phenotype, appeared to have lost t he VDJ_{H} rearrangement canonical to Ramos, and to have kept the J_{H} gene segments introduced by MMCT (FIGS. 3C and 3D). Others retained the canonical VDJ_{H}-rearrangement regardless of whether they contained surface Ig-positive cells (e.g., line RK2.2/5) or not (e.g., lines RK2.1/2, RK2.1/3, RK2.1/4 and RK2.1/5). These latter lines presumably lost surface Ig expression as a result of AICD-mediated mutation of the rearranged IgH or Igλ genes normally present in Ramos (see Sale et al., supra).

In order to convert Ramos cells into a practicable fusion partner, they were rendered resistant to G418 by stable transfection via electroporation (2×10⁷ cells, 0.3 ml RPMI medium, 0.25 kV, 960 μF, 0.4 cm electrode gap) with plasmid pCDNA3-RAG1 (see below). To transfer a “germline” chromosome 14 into these cells, microcells were made from A9-Hytk14 cells (Cuthbert et al., Cytogenet Cell Genet. 71(1):68-76 (1995)), fused to the G418-resistant Ramos cells using a protocol adapted from Hunt, Anal. Biochem., supra; Killary et al., Methods: A companion to Methods in Ezymology. 9:3-11 (1996) and Sanford et al., Som. Cell Mol. Genet. 13:279-284 (1987). Nine G418 (3 mg/ml, Sigma Aldrich, St Louis, Mich.) plus hygromycin (1000 U/ml, ICN Biochemicals, Aurora, Ohio) double-resistant colonies (originally designated Ram-14-c1 to Ram-14-c9 and later designated RK1.1 to RK1.9) were selected.

RK1.1 and RK1.2 cells were labeled with biotin-conjugated anti-IgM (Caltag, San Francisco, Calif.) then depleted of surface Ig-positive cells using streptavidin-conjugated magnetic beads (Miltenyi Biotech Inc., Auburn, Calif.) according to the manufacturer's instructions. Cells were plated at a density of ≦10 cells/well and the resulting colonies propagated as lines RK2.1/x and RK2.2/x where x=1 to 27.

DNA was extracted from cells using standard techniques and used in 35-cycle PCR reactions to detect IgH J gene segments J_{H}1 (with primer “A”, SEQ ID NO: 43, 5′-GGT CAC CGT CTC YTC AGG T-3′ and primer “B”, SEQ ID NO: 44, 5′-GAT ATC GAT ACC AGT AGC ACA GCC TCT G-3′), J_{H}2 (with primer “A” and primer “C”, SEQ ID NO: 45, 5′-CTG GAA TTC TGC AGG ACA CTC GAA TGG-3′), J_{H}3 (with primer “A” and primer “D”, SEQ ID NO: 46, 5′-TGC GGA TCC ACC TGA CTC TCC GAC TGT CC-3′), J_{H}4 (with primer “A” and primer “E”, SEQ ID NO: 47, 5′-CTA ACT AGT TGG GAC CCT CTC AGA CT-3′), J_{H}5 (with primer “A” and primer “F”, SEQ ID NO: 48, 5′-CCA TCT AGA CAG AGA CCT TCT GTC TCC G-3′) and J_{H}6 (with primer “A” and primer “G”, SEQ ID NO: 49, 5′-TGA GCG GGC CGC GGC CTC AAT TCC AGA CAC AT-3′), or the Ramos canonical VDJ_{H} rearrangement described in Sale et al. (Immunity, supra) (using primers jo1209, SEQ ID NO: 50, 5′-ATG AAA CAC CTG TGG TTC TTC CTC C-3′ and jo191, SEQ ID NO: 51, 5′-CGG GTA CCA ACC TGC AAT GCT CAG GA-3′). FIGS. 3A through 3D.

PCR products resolved by agarose gel electrophoresis were detected with ethidium bromide and are flanked by 100 bp DNA ladders. FIG. 3A. J_{H}1 to J_{H}4 are normally absent from Ramos, but were present in the double-resistant lines. One copy of J_{H}5 and two copies of J_{H}6 appeared to be already present in Ramos. Histograms representing surface Ig expression by viable (i.e. PI-excluding) cells are presented in FIG. 3B ((light line) Control Ramos cells cultured for 5 weeks after single-cell cloning, (bold dotted line) line RK1.1 after 9 weeks culture, (bold line) line RK1.2 after 9 weeks culture, (shaded) RK1.1 and RK1.2 cells 2 days after depletion for surface IgM-positive cells.) As shown in FIG. 3C, IgM production by limit clones (RK2.1/1 to 5 and RK2.2/1 to 5) was determined by flow cytometry after staining fixed and permeabilised cells with PE-conjugated anti-IgM ((shaded histograms) lines RK2.1/1 to 5 and RK2.2/1 to 4, (dotted white histogram) line RK2.2/5). The absence of the rearranged V(D)J_{H} gene segment in some surface Ig-depleted Ramos-glH sub-clones is shown in the agarose gel of FIG. 3D. PCR with (top) primers A and B was used to detect the J_{H}1 gene segment introduced by MMCT and with (bottom) primers jo1209 and jo191 to detect the VDJ sequence normally present on the VDJ-rearranged chromosome 14 carried by the parent Ramos cells. The source of the genomic DNA templates is indicated at the bottom of the figure.

### Example 3

### Induction of Gene Rearrangement in Reverted Lymphoma Cell Lines

(a) Cloning of cDNAs Encoding RAG-1, RAG-2 and TdT

In combination with housekeeping DNA repair pathways, the two RAG proteins, RAG-1 and RAG-2 (included in SEQ ID NOs: 2 and 4, respectively) are sufficient to carry out immunoglobulin V(D)J recombination in any cell while the TdT protein (included in SEQ ID NO: 6) is required for the insertion of random “N” nucleotides into the V(D)J junctions. These three proteins are referred to herein as “V(D)J recombinase.” Oettinger et al., Science 248:1517-1523 (1990) and Komori et al., Science 261:1171-1175 (1993).

DNA sequences encoding the human proteins RAG-1 and RAG-2 were PCR-amplified using DNA extracted from human cells as template and using PCR primers based on sequences in SEQ ID NOs: 1 and 3, respectively. A plasmid (MRE30) containing a human TdT cDNA sequence (SEQ ID NO: 5) was a gift of Micheal Ehrenstein and Michael Neuberger (MRC Laboratory of Molecular Biology, Cambridge, UK). See Sale and Neuberger, Immunity 9: 859-869 (1998). RAG-1, RAG-2 and TdT sequences were cloned into plasmid vectors that drive transient high level expression of introduced genes in human cells when introduced by transfection and/or adenoviral transduction.

(i) Expression of RAG1

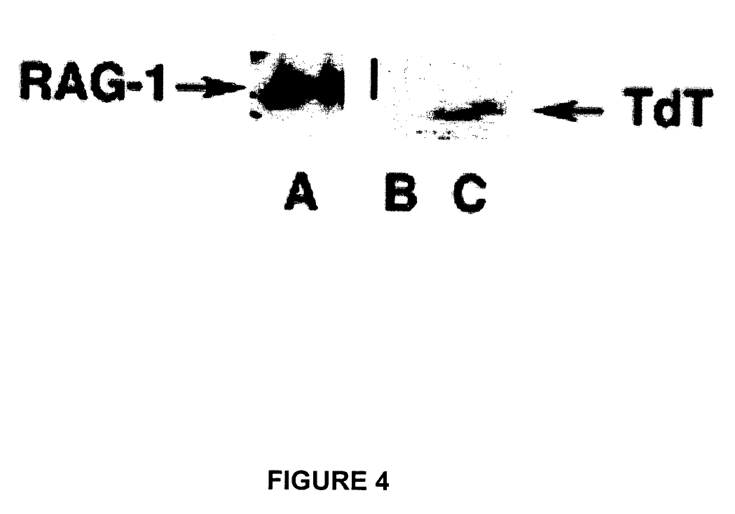

Western blotting of total cell extracts revealed that RAG1 protein was readily detectable in non-transfected Ramos cells. FIG. 4. Nonetheless, to ensure that RAG1 expression was stable, the RAG1 coding sequence was cloned by PCR amplification from human genomic DNA into the expression vector pcDNA3™ (Invitrogen Corporation, Carlsbad, Calif.) to produce plasmid pcDNA3-RAG1 (CJ087, SEQ ID NO: 40). This plasmid was stably introduced into Ramos cells by electroporation and selection with 3 mg/ml G418.

(ii) Western Blot Detection of RAG1 and TdT Proteins

Protein extracts of Ramos cells and Ramos cells transfected with plasmid MRE30 were prepared by boiling in SDS-PAGE loading buffer for 10 min. Extracts were resolved by SDS-PAGE and electro-blotted onto PVDF membrane (Bio-Rad Laboratories, Inc., Hercules, Calif.). Blots were probed using rabbit anti-human RAG1 (Santa Cruz Biotechnology Inc., Santa Cruz, Calif.) and mouse anti-human TdT (Dako Corporation, Carpinteria, Calif.)) antibodies. Bound antibodies were detected using peroxidase-conjugated anti-rabbit, or anti-mouse IgG (Santa Cruz Biotechnology), respectively, and chemiluminescense (Super Signal Substrate, Promega Corporation, Madison, Wis.).

(iii) Doxycycline-Regulated Expression of RAG2 and TdT

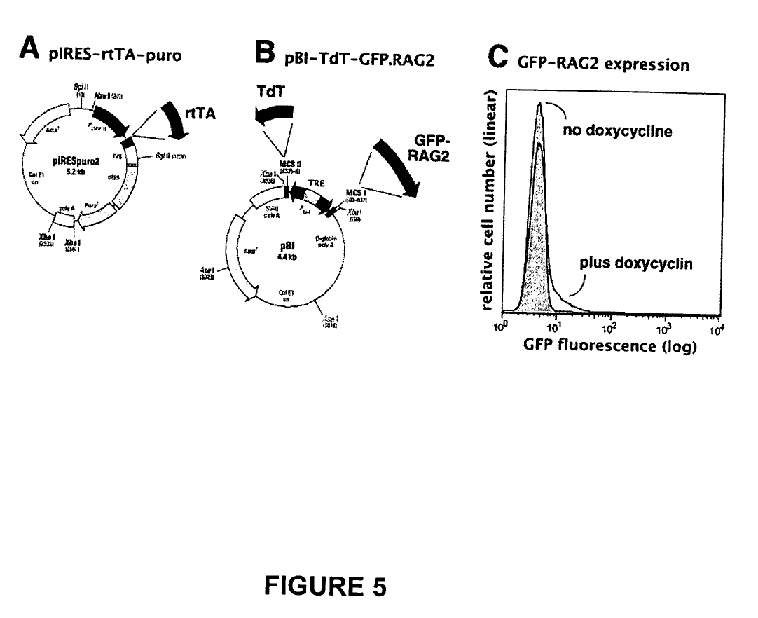

The reverse tetracycline-controlled transactivator (rtTA) sequence (CLONTECHniques XI(3):2-5 (1996); Gossen et al., Proc. Natl. Acad. Sci. USA 89:5547-5551 (1992); and Gossen et al., Science 268:1766-1769 (1995)) was sub-cloned from pRevTET-ON™ (BD Biosciences/Clontech, Palo Alto, Calif.) into the multi-cloning site of pIRESpuro2™ (BD Biosciences/Clontech) to produce plasmid pIRES-rtTA-puro (SEQ ID NO: 41, FIG. 5A). A sequence encoding mouse RAG2 N-terminally fused to enhanced green fluorescent protein (GFP), which was a gift of Dr Eugene Oltz (Vanderbilt University Medical School, Nashville, Tenn.) was sub-cloned into multi-cloning site I of the vector pBI (BD Biosciences/Clontech). A human TdT-encoding cDNA sequence was then sub-cloned from plasmid MRE30 into multi-cloning site R of the same plasmid to produce plasmid pBI-TdT-GFP.RAG2 (SEQ ID NO: 42). This plasmid (FIG. 5B) permits co-expression of TdT and GFP-RAG2 proteins in response to doxycycline in cells already expressing rtTA. Plasmids pBI-TdT-GFP.RAG2 and pIRES-rtTA-puro were co-transfected into RK2.1/4 cells and RK2.2/3 cells by electroporation and puromycin (10 μg/ml) resistant colonies were selected. Puromycin-resistant transfectants that exhibited enhanced GFP fluorescence in response to doxycycline (FIG. 5C) were designated RK5.7 and RK5.9, and RK6.6 and RK6.13, respectively.

(iii) Construction of Human Rag-2-Expressing Adenovirus Particles

Recombinant non-replicating adenovirus particles that could drive transient high level expression of RAG2, GFP, or GFP-RAG2 fusion protein were produced using the AdEasy™Adenoviral Vector system (Qbiogene, Inc., Carlsbad, Calif.). Briefly, sequences encoding RAG2 and the GFP-RAG2 fusion protein were separately cloned into the pShuttle-CMV plasmid (Stratagene, La Jolla, Calif.) to produce plasmids pShuttle-RAG2 and pShuttle-GFP-RAG2 (SEQ ID NOs: 52 and 53, respectively). The human RAG2 gene was amplified from Ramos genomic DNA by PCR using primers 5′-CAG ACA AGC TTC TAC GTA CCA TCA GA-3′ and 5′-GAT CAG AAT TCC TCA GTG AAG AAT A-3′ (SEQ ID NOs: 36 and 37, respectively). The resulting PCR product was cloned into the multi-cloning site of plasmid vector pShuttle-CMV (Stratagene, FIG. 2A). Similarly, the GFP-RAG2 sequence was sub-cloned into the multi-cloning site of plasmid vector pShuttle-CMV.

Full-length adenoviral DNAs carrying the RAG2 or GFP-RAG2 genes were created by homologous-recombination of pShuttle-CMV constructs with the pAdEasy-1 vector (Stratagene) in E. coli strain BJ5183 cells. FIG. 2B. By these methods, the plasmids pAdRAG2 and pAdGFP-RAG2 were produced. (SEQ ID NOs: 54 and 55, respectively). FIG. 2C. Purified pAdGFP (provided by Charles Bailey, Centenary Institute, and encoding a GFP-producing recombinant adenoviral genome, Shayakhmetov et al., J. Virol. 74(6):2567-2583 (2000)), pAdRAG2, and pAdGFP-RAG2 plasmids were digested with Pacd enzyme to liberate linear recombinant Adenoviral genomes and the linear DNAs were separately transfected into HEK293T cells to produce recombinant non-replicative adenovirus particles that could be used to infect Ramos cells. FIG. 2D.

(iv) Selection of Adenovirus-Susceptible Ramos Cells

Recombinant non-replicative adenovirus particles were produced from transiently-transfected HEK293T, amplified in HEK293T cultures and titred on AD293 cells following the protocols provided with the AdEasy™ Adenoviral Vector system kit (Stratagene). GFP-expressing recombinant adenoviral particles were used to transiently infect Ramos cells and cell lines derived from Ramos at MOI of 100. Two days after infection, cells expressing high levels of GFP were isolated using a FACStar Plus flow cytometric cell sorter (Beckton Dickenson). Sorted cells were later re-infected with GFP-adenovirus and the sort procedure repeated.

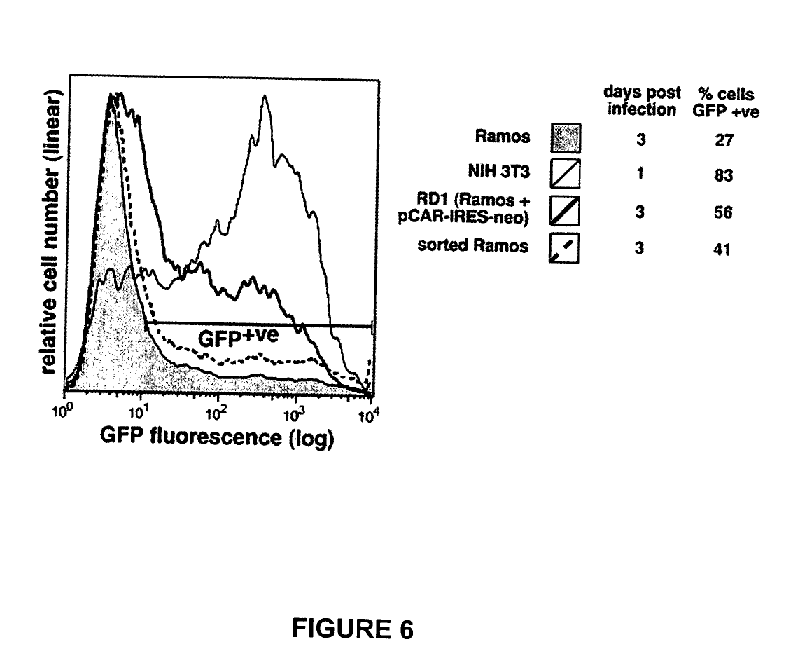

Initial experiments using recombinant GFP-expressing particles revealed that Ramos cells were relatively resistant to adenovirus infection when compared to NIH 3T3 cells. FIG. 6. Selection by flow cytometry for Ramos cells that expressed high levels of GFP during transient infection with non-replicating GFP-expressing a denovirus produced a cell line more susceptible to adenovirus infection than the starting cells. FIG. 6.

As an alternative approach to increasing susceptibility to infection, the human cocksackie-adenovirus receptor (CAR) cDNA was amplified by PCR from HEK293T cells using primers jo1272-CCA TCG ATG CCT ACC TGC AGC CGC CGC CC and jo1273-CGG GAT CCG AGG CTC TAT ACT ATA GAC (SEQ ID NOs: 61 and 62, respectively). The PCR product was sequenced and cloned into both pIRESneo and pIRESpuro2 plasmid vectors (BD Biosciences/Clontech) to generate plasmids pCAR-IRES-neo (pCJ124, SEQ ID NO: 63) and pCAR-IRES-puro (pCJ126, SEQ ID NO: 64).

Ramos cells were transfected with pCAR-IRES-neo DNA and G418 (3 mg/ml)-resistant colonies were subsequently screened for susceptibility to a denovirus infection by transiently infecting with the non-replicating GFP-adenovirus particles. The line displaying the highest levels of GFP expression 4 days later, as determined using a FACSCalibur flow cytometer (Becton, Dickinson and Company, Franklin Lakes, N.J.), was selected as the “adenovirus-susceptible” line RD1. FIG. 6.

(b) Transduction and Selection of Lymphoma Cells that Underwent New Rounds of V(D)J Recombination

Immunoglubulin gene-reverted Ramos lines (Ramos.proB, Ramos.glk, Ramos.glkH and Ramos.glH) are converted into adenovirus-susceptible lines by either of the methods described above. The RAG-2- or GFP-RAG2-expressing adenovirus particles are then used to infect (at MOI of 100:1) the immunoglobulin gene-reverted Ramos cells, as described above.

Transient expression of genes transduced into susceptible Ramos cells by infectious Adenovirus particles produced from pAdEasy-1 plasmid DNA occurs in greater than 40% of Ramos cells for up to five days after transduction. In the absence of selection for the retention of pAdEasy-1 DNA sequences, the circular adenoviral DNA (and hence RAG-2 expression) is lost as the cells divide. Expression of a functional V(D)J recombinase is detected by the appearance of surface immunoglobulin on some transfected cells as determined by staining with fluorescent anti-immunoglobulin antibodies and FACS.

(c) Fusion of RK2.2/3 Cells to Fresh Human B Cells

An alternative to inducing V(D)J recombination by introduction of a V(D)J recombinase is the fusion of a reverted pro-B cell-like or germline-like cells to fresh B cells.

(i) Isolation of Primary Human B Cells from Blood

Human blood (≧50 ml) is collected into heparin-coated tubes and diluted 1:1 with PBS at room temperature. Two volumes of blood are layered over one volume of Histopaqueo (Sigma-Aldrich, St. Louis, Mo.) or equivalent at room temperature and centrifuged at 800× g for 20 minutes at room temperature with no brake. The layer of white cells at the interface is recovered into a fresh tube and four volumes of PBS at 4° C. is mixed in. The cells are pelleted at 250× g for 5 minutes at 4° C., washed three more times with cold PBS, then counted. Alternatively, pooled buffy coats, human spleens, tonsils, lymph nodes, bone marrow or cord blood may be used as the source of B cells or plasmacytes.

Populations of primary human cells enriched for B cells and plasmacytes using, for instance, MACS® beads conjugated to anti-human CD19 or CD20 (Miltenyi Biotec, Germany) or using Dynabeads® conjugated to anti-human CD19 or CD20 (Dynal Biotech, Norway) plus DETACHaBEAD® CD19 or CD20 (Dynal Biotech, Norway) may be used for the following step. However, total peripheral blood nucleated cells, or splenic, tonsil, lymph node, bone marrow or cord blood cell populations depleted of red blood cells using, for instance Histopaque®, could also be used.

(ii) Fusion to RK2.2/3 Cells

A number of hybridoma fusion methodologies are readily available in the art. See, for example, “Antibodies: A Laboratory Manual” (ed. Harlow and Lane, Cold Spring Harbor Laboratory (1988)); “Monoclonal Antibodies: A Manual of Techniques (Zola et al., CRC Press (1987); “Rat Hybridomas and Rat Monoclonal Antibodies” (ed. Herve Bazin, CRC Press (1990); and Shirahata et al., Methods Cell Biol. 57:111-45 (1998). Most of these methodologies are suitable for generating fusions to Ramos or Ramos-derived cell lines.

The following exemplary fusion methodology may be employed to, for example fuse RK2.2/3 cells (“germline-IgH-reverted”) with human B cells. RK2.2/3 cells are grown to mid-log phase and mixed with freshly isolated human B cells at a ratio of 1:2. The mixed cells are pelletted at 250× g and washed once with serum-free RPMI medium. The cell pellet is resuspended, dropwise over 1 minute with gentle mixing at 37° C., in 0.8 ml polyethylene glycol (PEG, molecular weight 1300-1600) working solution (5 g PEG (Sigma-Aldrich, Catalogue No. P7777) plus 2.5 ml water) per 10⁷ RK2.2/3 cells. The tube is held at 37° C. for one minute while tapping gently to mix. RPMI medium (1 ml) at 37° C. is added over 1 minute with gentle mixing, then 20 ml RPMI at 37° C. is added over 4 minutes with gentle mixing. The cells are pelletted at 250× g for 7 minutes, resuspended in 20 ml culture medium (RPMI, supplemented with 10% (v/v) fetal calf serum and 50 μM 2-mercaptoethanol) and plated.

Alternatively, RK2.2/3 cells could be fused with primary human B cells by “electrofusion.” Zimmermann et al., Adv. Biotechnol. Processes 4:79-150 (1985); Finaz et al., Exp. Cell Res. 150(2):477-82 (1984); Vienken et al., FEBS Lett. 163(1):54-6 (1983); and Caude et al., Biochem. Biophys. Res. Commun. 114(2):663-9 (1983).

(iii) Selection for Fused Cells

48 hours after fusion, an additional 20 ml culture medium containing ganciclovir (5 μM) is added to the plated cells. This permits positive selection for RK2.2/3 cells that have replaced the Hytk-tagged “germline” chromosome 14 with a VDJ-rearranged chromosome 14 taken up from a primary human B cell.

(c) Purification of Surface-Immunoglobulin-Positive Cells

Two to twenty days after inducing expression of VDJ-recombinase or after fusing to fresh human B cells, viable cells are purified by layering on Ficoll-Paque® (Amersham Pharmaica Biotech AB, Uppsala, Sweden) at 800× g for 15 minutes then labeled with anti-human Igκ and anti-human Igλ Fab fragments conjugated to biotin. Cells that have successfully undergone new immunoglobulin gene V(D)J rearrangements are purified by virtue of their adherence to MACS strepatavidin MicroBeads as described by the manufacturer (Miltenyi Biotec, Bergisch Gladbach, Germany) and returned to culture in complete medium. The purified surface immunoglobulin-positive cells form the primary libraries of human monoclonal antibody-producing cells.

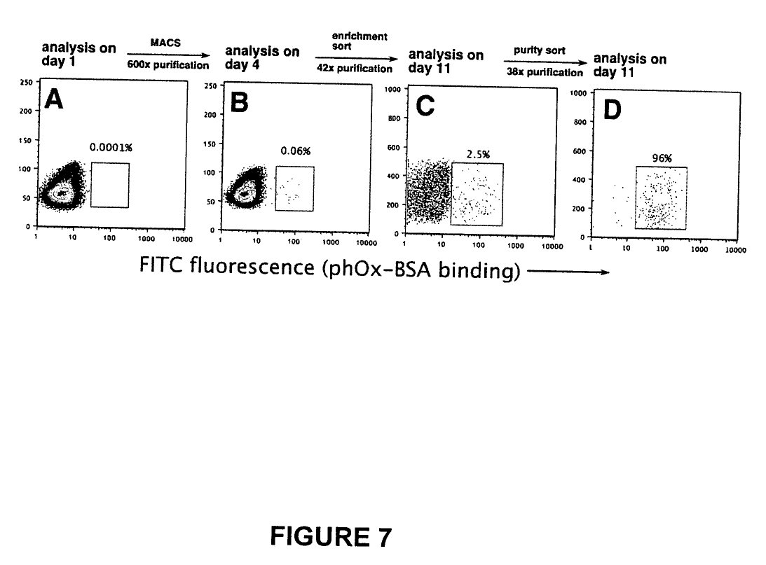

The libraries of V(D)J-rearranged lymphomas generated can be screened using combinations of panning and flow cytometry. Panning involves incubating Theramab library cells on ice in sterile medium with an excess of clinically relevant antigen conjugated to beads or to a plastic surface. The cells that bind to the antigen are recovered as a ‘positively selected library’. The positively selected library is returned to culture to expand the desired cell lines and the selection process repeated until the majority of cells in the library bind to the model antigen. Two or three rounds of this process can greatly enrich for cells expressing the appropriate antigen receptors. The degree of enrichment in each round can be assessed by surface-staining the selected cells with recombinant soluble antigen followed by flow cytometric analysis. Flow cytometry can also be used directly to purify individual monoclonal antibody-expressing clones of interest after initial panning. We have used panning and flow cytometric protocols to purify clones with specificity for the model antigen, phenoloxazolone (phOx, FIG. 7). A Ramos line (RCC64) specific for phOx was engineered by introducing plasmids encoding BCR heavy and light chains with V(D)J regions originally cloned from a mouse anti-phOx hybridoma. Kaartinen et al., J. Immunol. 130(2):937-45 (1983); Williams et al., Immunity 13:409-417 (2000).

The majority of RCC64 cells thus express a BCR consisting of IgM and Igκ chains carrying mouse V(D)J rearrangements conferring specificity for phOx. A culture of 10⁸ unmanipulated Ramos cells was “spiked” with 10² RCC64 cells, giving a frequency of one RCC64 cell for every 10⁶ unmanipulated Ramos cells. The cells were pelleted and incubated on ice in 5 ml PBA containing 0.2 μg/ml phOx₁₅-BSA-biotin conjugate for 30 minutes. The cells were pelleted twice to wash, then positively selected with streptavidin-conjugated MACS magnetic beads and an MS column according to the manufacturer's instructions (Miltenyi Biotec, Germany). The selected cells were mixed with 10⁴ “feeder” Ramos cells and returned to culture. After 3 days expansion in culture, an aliquot of cells was analysed for surface staining with phOx₁₅-BSA conjugate. It was thus evident that the first purifiaction step had achieved a 600-fold enrichment for phOx-binding cells. FIGS. 7A and 7B. Phenyloxazolone-binding cells were a further 42-fold enriched using a FACSVantage flow cytometer (Becton, Dickinson and Company, Franklin Lakes, N.J.) and low purity, high throughput settings. FIGS. 7B and 7C. This enriched population was immediately re-sorted for phOx-binding cells, but this time using high purity, low throughput settings to achieve a further 38-fold enrichment. This procedure gave a final purity of phOx-binding cells of 96% in less than two weeks—an enrichment of 9.6×10⁵-fold.

(d) Expansion and Storage of Selected Libraries

The antibody libraries are expanded by culturing the surface-immunoglobulin-positive cells in complete medium (described above) at densities of 2×10⁵ to 2×10⁶ cells/ml. Aliquots of 10⁸ cells are pelleted from culture medium at 250× g, resuspended in 2 ml culture medium+10% DMSO and slowly chilled to the temperature of liquid nitrogen using standard techniques. Bonifacino et al., “Current Protocols in Cell Biology” (John Wiley & Sons Inc., 1999). The library aliquots are cryo-preserved in liquid nitrogen.

### Example 4

### Isolation of Fully Human Monoclonal Antibodies to Human Rhesus Factor (Blood Group Antigen RhD)

The RhD factor or rhesus factor is an erythrocyte surface antigen. RhD incompatibility is a condition that occurs when a woman of RhD-negative blood type (˜15% of the population) is exposed to RhD-positive blood cells and subsequently produces circulating antibodies to RhD factor. RhD incompatibility commonly occurs when a pregnant RhD-negative woman is exposed to RhD-positive fetal erythrocytes, and is potentially fatal for the fetus. Maternal exposure to the fetal blood cells can occur as a result of spontaneous or induced abortion, trauma, invasive obstetrical procedures, or delivery. This example demonstrates the preparation and isolation of cells that express fully-human monoclonal antibodies having RhD antigen specificity.

(a) Production of the Selecting Antigens

Using cDNA synthesized from human reticulocyte RNA as template, and using primers based on Genbank accession L08429 (SEQ ID NO: 38), the coding region for the human RhD blood group antigen is cloned by PCR into the pcDNA3.1 plasmid vector. CHO cells are stably transfected with the RhD construct by electroporation (using 2×10⁷ cells, a 4 mM electrode gap, 0.25 kV, and 960 μF) and selected for by G418. RhD^{+ve} CHO clones (identified using anti-RhD monoclonal antibodies; Perera et al., Transfusion 40:846-55 (2000)) are expanded in culture. RhD^{+ve}-erythrocytes and RhD^{-ve}-erythrocytes separately pooled from multiple donors (provided by the Red Cross) are fixed with 2% paraformaldehyde, reacted with sulfo-NHS-biotin (Pierce, Rockford Ill., USA) to label the cells with conjugated biotin, then washed three times with phosphate buffered saline (PBS).

(b) Selecting for Cells that Bind the RhD Antigen

Libraries of V(D)J-rearranged lymphoma cells are incubated on ice in sterile medium with an excess of RhD-negative human blood cells. The lymphoma cells that do not bind to the RhD-negative blood cells are recovered as a “negatively-selected library”—these are lymphomas that do not make non-specific antibodies against blood cells. The negatively selected lymphoma library is incubated with RhD-positive human blood cells, and lymphoma cells that bind to the RhD-positive cells (“positively-selected library”) are recovered. The positively selected library is returned to culture to expand the desired lymphomas, and the selection process repeated until the majority of lymphomas in the library bind RhD-positive cells and not-RhD-negative cells.

Two or three rounds of this process may be required to enrich for cells expressing RhD factor-specific immunoglobulins. Surface-staining the selected Burkitt's lymphoma cells with recombinant soluble RhD factor followed by FACS analysis will be used to assess the degree of enrichment in each round. Similarly, FACS may be used to purify useful individual monoclonal antibody-expressing clones. Details of these procedures are presented below.

An aliquot (10⁸ cells) of the Ramos monoclonal antibody library is thawed quickly at 37° C., washed in complete medium and cultured for 24 hour to recover from thawing. The cells are resuspended in 5 ml ice-cold complete medium. Fixed biotinylated RhD^{-ve}-erythrocytes (10¹⁰) are resuspended in another 5 ml ice-cold complete medium. The two cell suspensions are combined and gently mixed for 20 min at 20° C. The cell suspension is divided between two 15 ml centrifuge tubes, underlayed with an equal volume of Ficoll-Paque® (Amersham Pharmaica Biotech AB, Uppsala, Sweden) and centrifuged for 15 min at 800× g at 20° C. The cells floating on top of the high-density solution are recovered, washed with ice-cold PBS+0.5% bovine serum albumin (PB), and resuspended in ice-cold PB to 10⁸ cells/ml. Dead cells and cells that bind to non-RhD erythrocyte antigens or to biotin are depleted from the library by this procedure.

MACS strepatavidin MicroBeads (Miltenyi Biotec, Bergisch Gladbach, Germany) are added (100 μl beads/10⁸ lymphoma cells) and the suspension mixed gently at 10° C. After 15 min, the suspension is passed through a MACS separation column L (Miltenyi Biotec) under gravity at 4° C. in the presence of a strong magnetic field generated by a MidiMACS apparatus (Miltenyi Biotec). The column is washed three times with 3 ml of ice-cold PB to elute unbound cells, which are then pelleted and resuspended to 2×10⁷ cells/ml in ice-cold PB+0.02% azide (PBA). Cells that bind directly to MACS strepatavidin MicroBeads are depleted by this procedure.

An equal volume of biotinylated, fixed RhD^{+ve}-erythrocytes at 2×10⁶ cells/ml in ice-cold PBA is added to the depleted library cell suspension. The cell suspension is gently mixed for 30 min at 10° C. MACS streptavidin MicroBeads are added (10 μl per 10⁶ cells) and the cell/bead suspension gently mixed at 10° C. for 20 min. The cell suspension is then passed through a MACS separation column L under gravity at 4° C. as above. The column is washed three times with 3 ml of ice-cold PBA to elute unbound cells, removed from the MidiMACS apparatus, and the cells that were retained in the magnetic field by virtue of binding directly or indirectly to the MACS beads are eluted in 5 ml of ice-cold culture medium. These cells are washed 2 times with 5 ml of ice-cold culture medium and returned to culture at 5×10⁵ cells/ml with the addition of Ramos feeder cells if necessary. These cultures are enriched for clones that bind the RhD antigen.

(c) Testing for Enrichment of Anti-RhD Antigen Clones

Once the culture has re-grown to 10⁸ cells, half of the cells are removed from culture and treated with pepsin at pH 4 to release F(ab′)₂ fragments from the surface of the cells, and a dilution series of the pepsin supernatant is made in culture medium lacking sodium bicarbonate, but supplemented with 10 mM HEPES, pH 7.4. Bonifacino et al, supra. Monolayers of RhD^{+ve}-CHO cells and normal CHO cells are grown in 96-well plates. The monolayers are washed, then incubated for 30 min at 4° C. with the F(ab′)₂ dilution series. The monolayers are washed again and F(ab′)₂-binding quantified using anti-human light chain antibodies and standard ELISA techniques. The ELISA signal produced in the normal CHO cell plates is subtracted from the signal produced in the RhD^{+ve}-CHO cell plates. Successful enrichment by the selection procedure is indicated by an increase in the titre of anti-RhD F(ab′)₂ fragments/μg IgL chains compared to the unselected library or to the previous round of selection. The selection procedure is repeated until the rise in titre of anti-RhD F(ab′)2 fragments plateaus.

### Example 5

### Isolation of Individual RhD-Binding Clones

RhD^{+ve}-erythrocytes are directly labelled with FITC. Colligan et al., supra. Biotin-labelled RhD ve-erythrocytes are incubated in ice-cold PB with phycoerythrin (PE)-conjugated streptavidin (Caltag Laboratories, Burlingame, Calif., USA) for 20 min, and washed 3× with PB. 10⁷ cells from the pool of RhD-binding enriched library cells are mixed with 10⁷ FITC^{+ve}RhD^{+ve}-erythrocytes and 10⁸ PE^{+ve}RhD^{-ve}-erythrocytes in 1 ml ice-cold PB and incubated on ice for 30 min. Individual library cells that have bound to FITC^{+ve} erythrocytes, but not PE^{+ve} erythrocytes are purified by fluoresence-activated cell sorting (FACS), and deposited into 96-well culture plate wells containing sterile medium previously conditioned by growing Ramos cells up to a density of about 5×10⁵ cells per ml prior to being re-sterilized by 0.2 mm filtration.

Individual clones are grown to ≧2×10⁶ cells, and F(ab′)₂ fragments prepared from 10⁶ cells and titred as described in Example 4. Clones producing the highest titre F(ab′)₂ are expanded and frozen down as described in Example 3.

### Example 6

### Selecting Higher Affinity Anti-RhD Clones

Anti-RhD Ramos clones are maintained in culture at 2×10⁵ to 5×10⁶ cells/ml for 6 weeks to allow the rearranged immunoglobulin V regions to mutate. Sale et al., supra. Anti-RhD cells are purified by FACS as described in Example 5, except that the ratio of anti-RhD cells to RhD^{+ve}-erythrocytes during labeling of the anti-RhD cells is 20:1, and labeling performed on ice for only 5 minutes.

Alternatively, the cultured cells are labelled with FITC^{+ve}RhD^{+ve}-erythrocytes and PE^{+ve}RhD^{-ve}-erythrocytes as in Example 5, and are additionally labelled with APC-conjugated anti-human Igκ and APC-conjugated anti-human Igλ antibodies. Anti-RhD cells are purified by FACS as described in Example 5, but with additional selection for cells that exhibit the highest ratios of FITC:APC fluorescence.

After expanding sorted clones as described in Example 5, clones producing the highest titer of anti-RhD F(ab′)₂ₘ are expanded and frozen down as described in Example 3.

### Example 7

### Producing IgG Anti-RhD Clones by Inducing Isotype Switching

Selected anti-RhD clones are cultured at 2×10⁵ to 5×10⁶ cells/ml with recombinant human CD40L, IL4 and IL10 for 2 weeks. Cells that have switched isotype to IgG are identified and purified by FACS after staining with FITC-anti-human IgG antibodies. These cells are expanded as described in Example 5 and their expression of IgG anti-RhD antibodies confirmed.

### Example 8

### “Fixing” the Antibody Secreted by Selected Clones

The human Blimp-1 cDNA is amplified from multiple myeloma cDNA by PCR (using primers jo1289-CGG ATA TCG CTG CCC CCA AGT GTA ACT C (SEQ ID NO: 65), and jo1290-TAA AGC GGC CGC TTA CTT ATC GTC GTC ATC CTT GTA ATC AGG ATC CAT TGG TTC AAC TGT CTC (SEQ ID NO: 66)) and cloned into the plasmid pIRESbleo (Invitrogen) to produce plasmid pBlimp1-IRES-bleo (SEQ ID NO: 67).

The IgG^{+ve} anti-RhD clones selected in Example 7 are transfected with pBlimp1-IRES-bleo DNA and cells resistant to 2.5 mg/ml Zeocin™ (Invitrogen) are selected. Blimp-1 expression supresses expression of AICD and thus prevents further Ig gene mutation, and also increases levels of antibody secretion. Shaffer et al., Immunity 17:51-62 (2002). Zeocin™-resistant colonies are screened by ELISA for Ig secretion, and the colonies secreting the highest levels of Ig are selected.

### Example 9

### Producing Anti-RhD IgG Antibodies

Blimp-1^{+ve} IgG^{+ve} anti-RhD clones selected in Example 8 are grown in CellMax® hybridoma culture apparatus as recommended by the manufacturer (Spectrum Laboratories, Rancho Dominguez, Calif., USA) to produce culture supernatants containing secreted IgG, and the antibody is purified using HiTrap rProtein A columns as recommended by the manufacturer (Amersham Pharmacia Biotech). Clones that secrete sub-optimal amounts of IgG are fused to mouse NS0 cells using standard techniques to produce hybridomas. Bonifacino et al., supra. Hybridoma clones are screened for titers of secreted anti-RhD IgG1 using the ELISA described in Example 4. Alternatively, the VDJ_{H}- and VJ_{L}-rearrangements in lines isolated from the libraries are sub-cloned by PCR into antibody-expression vectors and stably introduced into COS cells.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

What is claimed is:

1. A vector for reverting cell lines to a pro-B cell-like state or to a germline-like state, said vector comprising one or more immunoglobulin regions selected from the group consisting of V regions, D regions, and J regions, a 5′ flanking region operably linked 5′ to a 5′-most region and a 3′ flanking region operably linked 3′ to a 3′-most region wherein said 5′ and 3′ flanking regions are capable of facilitating homologous recombination of said immunoglobulin regions into a cell having a V(D)J rearranged immunoglobulin gene.

2. The vector of claim 1 wherein said vector comprises one or more fused DJ regions.

3. The vector of claim wherein said vector comprises two, three, four, five or more fused DJ regions.

4. The vector of claim 1 wherein said vector comprises six fused DJ regions.

5. The vector of claim 1 further comprising a 5′ flanking region and a 3′ flanking region.

6. The vector of claim 1 further comprising a selectable marker operably linked 3′ to said 5′ flanking region and 5′ to said 5′-most region.

7. The vector of claim 6 wherein said selectable marker is operably linked 5′ to the 5′-most DJH region.

8. The vector of claim 6 wherein said selectable marker is Eco-gpt.

9. A vector comprising a polynucleotide sequence encoding a recombination-promoting protein, or functional fragment, derivative, or variant thereof, selected from the group consisting of RAG-1 and RAG-2.

10. The vector of claim 9 wherein said vector is a plasmid vector.

11. The vector of claim 9 wherein said vector is a viral vector.

12. The vector of claim 11 wherein said viral vector is an adenoviral vector.

13. The vector of claim 9 selected from the group consisting of pCDNA3-RAG1 (SEQ ID NO: 40), pBI-TdT-GFP.RAG2 (SEQ ID NO: 42), pShuttle-RAG2 (SEQ ID NO: 52), pShuttle-GFP-RAG2 (SEQ ID NO: 53), pAdEasy-RAG2 (SEQ ID NO: 54), pAdEasy.1-GFP-RAG2 (SEQ ID NO: 55), and pAdEasy.2-GFP-RAG2 (SEQ ID NO: 56).

14. A method for generating immunoglobulin heavy and/or light chains, said method comprising the steps of:
(a) reverting a cell comprising a V(D)J rearranged immunoglobulin region by introducing into said cell a polynucleotide encoding V, D, and/or J regions of an immunoglobulin heavy and/or light chain, or fragment thereof, wherein said V, D, and/or J regions replace said V(D)J rearranged immunoglobulin region such that the introduced V, D, and/or J regions are in a pro-B cell-like or a germline-like state; and
(b) expressing in said reverted cell a polynucleotide sequence encoding a recombination-facilitating protein, or functional fragment, derivative or variant thereof, for a time and under conditions sufficient to induce rearrangement of the V, D, and/or J regions, wherein rearrangement of the V, D, and/or J regions facilitates expression of an immunoglobulin heavy and/or light chain.

15. A method for generating immunoglobulin heavy chains, said method comprising the steps of:
(a) reverting a cell comprising a V(D)J rearranged immunoglobulin region by introducing into said cell a polynucleotide encoding fused DJ regions of an immunoglobulin heavy chain, wherein said DJ regions replace said V(D)J rearranged immunoglobulin region such that the introduced fused DJ regions are in a pro-B cell-like state; and
(b) expressing in the reverted cell a polynucleotide sequence encoding a recombination-facilitating protein, or functional fragment thereof, for a time and under conditions sufficient to induce rearrangement of the germline V regions in the reverted cell with the introduced fused DJ regions,
wherein rearrangement of the V and fused DJ regions facilitates expression of an immunoglobulin heavy chain.

16. A method for generating immunoglobulin light chains, said method comprising the steps of:
(a) reverting a cell comprising a V(D)J rearranged immunoglobulin region by introducing into said cell a polynucleotide encoding J regions of an immunoglobulin light chain, wherein said J regions replace said V(D)J rearranged immunoglobulin region such that the introduced J regions are in a pro-B cell-like or a germline-like state; and
(b) expressing in the reverted cell a polynucleotide sequence encoding a recombination-facilitating protein, or functional fragment thereof, for a time and under conditions sufficient to induce rearrangement of the germline V regions in the reverted cell with the introduced J regions, wherein rearrangement of the V and J regions facilitates expression of an immunoglobulin light chain.

17. A method for generating libraries of cells that produce an array of immunoglobulins wherein each immunoglobulin exhibits a particular antigen specificity, said method comprising the steps of:
(a) providing a cell having a V(D)J rearranged immunoglobulin heavy and/or light chain region;
(b) introducing into the cell a polynucleotide encoding V, D, and/or J regions of an immunoglobulin heavy and/or light chain, or fragment thereof, wherein said V, D, and/or J regions replace said V(D)J rearranged immunoglobulin heavy and/or light chain region such that the introduced V, D, and/or J regions are in a pro-B cell-like or a germline-like state;
(c) culturing the cell under suitable conditions to generate a reverted cell population each member of which population comprises V, D, and/or J regions in a pro-B cell-like or a germline-like state;
(d) introducing into cells of the reverted cell population a polynucleotide sequence encoding a recombination-facilitating protein, or functional fragment, derivative or variant thereof; and
(e) culturing the resulting population of cells expressing the recombination-facilitating protein for a time and under conditions sufficient to induce rearrangement of the pro-B cell-like or germline-like V, D, and/or J regions, wherein rearrangement of the V, D, and/or J regions facilitates expression of an immunoglobulin heavy and/or light chain having a particular antigen specificity.

18. A method for identifying in a cell an immunoglobulin having a desired antigen specificity, said method comprising the steps of:
(a) reverting a cell comprising a V(D)J rearranged immunoglobulin region by introducing into said cell a polynucleotide encoding V, D, and/or J regions of an immunoglobulin heavy and/or light chain, or fragment thereof, wherein said V, D, and/or J regions replace said V(D)J rearranged immunoglobulin region such that the introduced V, D, and/or J regions are in a pro-B cell-like or a germline-like state;
(b) expressing in the reverted cell a polynucleotide sequence encoding a recombination-facilitating protein, or functional fragment, derivative, or variant thereof, for a time and under conditions sufficient to induce rearrangement of the pro-B cell-like or germline-like V, D, and/or J regions, wherein rearrangement of the V, D, and/or J regions facilitates expression of an immunoglobulin heavy and/or light chain; and
(c) screening the resulting V, D, and/or J region rearranged cells for an immunoglobulin having the desired antigen specificity.

19. A method for generating cell lines capable of producing immunoglobulins having a desired specificity, said method comprising the step of reverting a cell comprising a V(D)J rearranged immunoglobulin region by introducing into said cell a polynucleotide encoding V, D, a nd/or J regions of an immunoglobulin heavy and/or light chain gene, or fragment thereof, wherein said V, D, and/or J regions replace said V(D)J rearranged immunoglobulin region such that the introduced V, D, and/or J regions are in a pro-B cell-like or a germline-like state.

20. A method for generating cell lines capable of producing immunoglobulins having a desired specificity, said method comprising the step of reverting a cell comprising a V(D)J rearranged immunoglobulin region by introducing into said cell a polynucleotide encoding one or more fused DJ regions of an immunoglobulin heavy chain, wherein said fused DJ regions replace said V(D)J rearranged immunoglobulin region such that the introduced fused DJ regions are in a pro-B cell-like state.

21. The method of claim 20 wherein said introduced polynucleotide comprises two or more fused DJ regions.

22. The method of claim 20 wherein said introduced polynucleotide comprises at least three, four, or five fused DJ regions.

23. The method of claim 20 wherein said introduced polynucleotide comprises six fused DJ regions.

24. A method for producing immunoglobulins having a particular affinity or specificity for a target molecule, said method comprising the steps of:
(a) generating a reverted lymphoma cell line capable of producing immunoglobulins;
(b) expressing a polynucleotide encoding a recombination-facilitating protein, or functional fragment, derivative, or variant thereof, in the reverted lymphoma cell line for a time and under conditions sufficient to induce a rearrangement of the genes encoding the immunoglobulins which facilitates the generation of a library of lymphoma cells which produce an array of immunoglobulins wherein each immunoglobulin exhibits a particular affinity or specificity; and
(b) screening for immunoglobulins having a desired affinity or specificity.

25. A method for producing libraries of human monoclonal antibody-producing lymphoma cells, said method comprising the step of expressing in a reverted human lymphoma cell a polynucleotide encoding a recombination-facilitating protein for a time and under conditions sufficient to induce rearrangement of genes encoding human antibodies in the reverted lymphoma cells.

26. A method for generating a library of lymphoma cell lines of human origin, each human lymphoma cell line capable of producing fully-human immunoglobulins of a particular specificity, said method comprising the steps of:
(a) reverting a human lymphoma cell line comprising a V(D)J rearranged immunoglobulin region by introducing a polynucleotide encoding human V, D, and/or J regions of human immunoglobulin heavy and/or light chains into said human lymphoma cell line, wherein said V, D, and/or J regions replace said V(D)J rearranged immunoglobulin region such that the introduced V, D, and/or J regions are in a pro-B cell-like or germline-like state; and
(b) expressing in the reverted human lymphoma cell a polynucleotide sequence encoding a human recombination-facilitating protein, or a functional fragment, derivative or variant thereof, for a time and under conditions sufficient to facilitate rearrangement of immunoglobulin-encoding genes thus generating a library of human lymphoma cells each producing a fully-human immunoglobulin of a particular specificity.

27. A method for generating a library of lymphoma cell lines of human origin, each human lymphoma cell line capable of producing fully-human immunoglobulin heavy chains of a particular specificity, said method comprising the steps of:
(a) reverting a human lymphoma cell line comprising a V(D)J rearranged immunoglobulin region in by introducing a polynucleotide encoding fused DJ regions of a human immunoglobulin heavy chain into said cell line, wherein said DJ regions replace said V(D)J rearranged immunoglobulin region such that the introduced fused DJ regions are in a pro-B cell-like state;
(b) expressing in the reverted human cell a polynucleotide sequence encoding a human recombination-facilitating protein, or functional fragment, derivative or variant thereof, for a time and under conditions sufficient to induce rearrangement of the human lymphoma cell line germline V regions with the introduced fused DJ regions, wherein rearrangement of the V and fused DJ regions facilitates expression of a fully-human immunoglobulin heavy chain.

28. A method for generating a library of lymphoma cell lines of human origin, each human lymphoma cell line capable of producing fully-human immunoglobulin light chains of a particular specificity, said method comprising the steps of:
(a) reverting a human lymphoma cell comprising a V(D)J rearranged immunoglobulin region in by introducing a polynucleotide encoding J regions of a human immunoglobulin light chain into said human lymphoma cell, wherein said J regions replace said V(D)J rearranged immunoglobulin region such that the introduced J regions are in a pro-B cell-like or a germline-like state;
(b) expressing in the reverted human lymphoma cell line a polynucleotide sequence encoding a human recombination-facilitating protein, or functional fragment, derivative or variant thereof, for a time and under conditions sufficient to induce rearrangement of the germline V regions with the introduced J regions, wherein rearrangement of the V and J regions facilitates expression of a fully-human immunoglobulin light chain.

29. The method of any one of claims 14-23 and 26-28 wherein reversion of cells to a pro-B cell-like state is achieved by introducing a vector comprising a polynucleotide encoding fused DJ regions of an immunoglobulin heavy chain, or fragment thereof.

30. The method of any one of claims 14, 16-20, 26, and 28 wherein reversion of cells to a germline-like state is achieved by introducing a vector comprising a polynucleotide encoding V, D, and/or J regions of immunoglobulin heavy and/or light chains, or fragments thereof, wherein the V, D, and/or J regions are assembled in the vector in a germline-like state.

31. The method of any one of claims 14, 16-20, 26, and 28 wherein reversion of cells to a germline-like state is achieved by introducing a chromosome, or substantial portion thereof, wherein said chromosome encodes a complete germline heavy or light chain antibody repertoire or substantial portion thereof.

32. The method of claim 31 wherein said chromosome is a human chromosome selected from the group consisting of chromosomes 2, 14, and 22.

33. The method of claim 31 wherein said chromosome replaces the corresponding endogenous V(D)J rearranged chromosome.

34. The method of claim 31 wherein said chromosome is introduced into said V(D)J rearranged cell by a methodology selected from the group consisting of microcell-mediated chromosome transfer, T cell-fusion, micro-injection, and yeast protoplast fusion.

35. The method of claim 31 wherein said cells comprising V(D)J rearranged immunoglobulin regions are reverted to a germline-like configuration by fusing a B cell line with precursor B cells isolated from human bone marrow or cord blood.

36. The method of claim 31 wherein said cell is reverted to a germline-like configuration by fusing a B cell line with T cells isolated from human blood.

37. The method of claim 31 wherein said cell is are reverted to a germline-like configuration by fusing B cell lines with rodent/human somatic cell hybrids carrying a single human chromosome.

38. The method of any one of claims 14-16 and 24 wherein the immunoglobulin heavy and/or light chain regions are human immunoglobulin heavy and/or light chain regions.

39. The method of any one of claims 14-16 and 24 wherein said immunoglobulin is from an animal selected from the group consisting of primate, sheep, pig, cow, horse, donkey, poultry, rabbit, mouse, rat, guinea pig, hamster, dog, and cat.

40. The method of any one of claims 14-18, 24-28 wherein said recombination-facilitating protein, or functional fragment, derivative, or variant thereof, is selected from the group consisting of RAG-1 and RAG-2.

41. The method of any one of claims 14-18, 24-28 wherein said polynucleotide encoding said recombination-facilitating protein, or functional fragment, derivative, or variant thereof, is introduced into said cell on an adenoviral vector.

42. The method of claim 41 wherein said recombination-facilitating protein, or functional fragment, derivative, or variant thereof, is from an animal selected from the group consisting of primates, livestock animals, laboratory test animals, companion animals, avian animals, reptilian animals, amphibian animals, and aquatic animals.

43. The method of claim 41 wherein said recombination-facilitating protein is human RAG-1 as depicted herein in SEQ ID NO: 2 or a functional fragment, derivative, or variant thereof.

44. The method of claim 41 wherein said recombination-facilitating protein is human RAG-2 as depicted herein in SEQ ID NO: 4 or a functional fragment, derivative, or variant thereof.

45. The method of claim 43 or 44 wherein said recombination-facilitating protein, or functional fragment, derivative or variant thereof, is expressed transiently for a time and under conditions sufficient to achieve recombination in said reverted cell.

46. The method of claim 43 or 44 wherein said recombination-facilitating protein, or functional fragment, derivative or variant thereof, is expressed constitutively and wherein expression is under the control of an inducible transcriptional promoter.

47. The method of any one of claims 15-29 wherein said immunoglobulin-producing cells are selected from the group consisting of lymphocytes, lymphocyte cell lines, B lymphocytes, B lymphocyte cell lines, B cell lymphoma cells, and B cell lymphoma cell lines.

48. The method of any one of claims 15-29 wherein said immunoglobulin-producing cells are a human B cell line.

49. The method of any one of claims 15-29 wherein said cells are generated from patients with Burkitt's lymphoma.

50. The method of any one of claims 15-29 wherein said cells are selected from the group consisting of Ramos, Ramos sub-line 2G6, Burkitt's lymphoma cell line BL2, Burkitt's lymphoma cell line BL16, and BL16 sub-line CL-01.

51. An immunoglobulin heavy and/or light chain generated by any one of the methods of any one of claims 15-17 and 25.

52. An immunoglobulin fragment generated from the immunoglobulin heavy and/or light chain of claim 51 wherein said immunoglobulin fragment is selected from the group consisting of an Fab, an F(ab′)2, an Fc, and scFv fragment.

53. The method of any one of claims 18, 20-24, and 26-29 further comprising the step of treating said cells expressing said immunoglobulin heavy and/or light chains, or fragments thereof, with an agonist to induce switching from a first antibody isotype to a second antibody isotype wherein said first antibody isotype is selected from the group consisting of IgM, IgD, IgG1, IgG2, IgG3, IgG4, IgE, IgA1 and IgA2 and wherein said second antibody isotype is selected from the group consisting of IgD, IgG1, IgG2, IgG3, IgG4, IgE, IgA1 and IgA2.

54. The method of claim 53 wherein said agonist is selected from the group consisting of a ligand for CD40, a ligand for the B cell receptor, a B cell mitogen, a cytokines, bacterial DNA, a synthetic oligonucleotide containing unmethylated CpG dinucleotides, anti-CD19, and anti-CD21.

55. The method of any one of claims 18, 20-24, and 25-29 further comprising the step of subjecting to mutagenesis said cells or libraries of cells expressing one or more immunoglobulins, or fragments thereof.

56. The method of claim 55 wherein said mutagenesis step comprises introducing into the cell and/or library of cells a polynucleotide encoding activation-induced cytidine deaminase (AICD) or a functional fragment, derivative or variant thereof.

57. The method of claim 55 wherein said mutagenesis step is achieved by endogenous expression of an activation-induced cytidine deaminase (AICD).

58. The method of claim 57 wherein said endogenous expression of said activation-induced cytidine deaminase (AICD) is constitutive.

59. The method of claim 57 wherein said endogenous expression of said activation-induced cytidine deaminase (AICD) is induced.

60. The method of claim 58 further comprising the step of introducing into the cells and/or libraries of cells a polynucleotide encoding Terminal Deoxynucleotidyltransferase (TdT) or a functional fragment, derivative or variant thereof.
